(19) United States
(12) Patent Application Publication
(10) Pub. No.: US 2025/0188493 A1
(43) Pub. Date: Jun. 12, 2025

(54) RECOMBINANT VIRAL VECTOR AND USES THEREOF

(71) Applicant: University of Miami, Miami, FL (US)

(72) Inventor: GLEN N. BARBER, MIAMI, FL (US)

(73) Assignee: UNIVERSITY OF MIAMI, MIAMI, FL (US)

(21) Appl. No.: 19/030,652

(22) Filed: Jan. 17, 2025

Related U.S. Application Data
(63) Continuation of application No. 17/041,435, filed on Sep. 24, 2020.
(60) Provisional application No. 62/648,096, filed on Mar. 26, 2018.

(51) Int. Cl. C12N 15/86; A61P 35/00; C07K 14/705; C12N 9/12

(52) U.S. Cl. CPC C12N 15/86; A61P 35/00; C07K 14/705; C12N 9/1241; C12N 2760/20232; C12N 2760/20243; C12N 2760/20262

(57) ABSTRACT

The present disclosure relates, in general, to recombinant viral vectors that stimulate STING (STimulator of INterferon Genes) activity and increase activity of immune cells.

### CROSS-REFERENCE TO RELATED APPLICATIONS

This application is a continuation of (i) U.S. 371 NationalPphase application Ser. No. 17/041,435, filed Sep. 24, 2020, which claims the priority benefit of (ii) U.S. National Phase Application of PCT/US2019/024039, filed Mar. 26, 2019, which claims the priority benefit of (iii) U.S. Provisional Application No. 62/648,096, filed Mar. 26, 2018, the disclosure of (i)-(iii) are incorporated herein by reference in their entireties and for all purposes.

### STATEMENT AS TO FEDERALLY SPONSORED RESEARCH

This invention was made with government support under grant number CA194404 awarded by the National Institute of Health (NIH). The government has certain rights in the invention.

### FIELD OF THE INVENTION

The present disclosure relates, in general, to recombinant viral vectors that stimulate STING (STimulator of INterferon Genes) activity and increase activity of immune cells.

### INCORPORATION BY REFERENCE OF MATERIALS SUBMITTED ELECTRONICALLY

The Sequence Listing written in file STNG-01004US2_ST26.xml, created Jan. 17, 2025, 27,490 bytes, machine format IBM-PC, MS-Windows operating system, is hereby incorporated by reference.

### BACKGROUND

Stimulator of interferon (IFN) genes (STING) is a key mediator in the immune response to cytoplasmic DNA sensed by cyclic GMP-AMP (cGAMP) synthase (cGAS). After synthesis by cGAS, cGAMP acts as a second messenger activating STING in the cell harboring cytoplasmic DNA but also in adjacent cells through gap junction transfer. STING appears to be an essential component in the recruitment of immune cells to the tumor microenvironment, which is paramount to immune clearance of the tumor.

It has been shown that mice lacking the innate immune regulator STING (stimulator of interferon genes) are also sensitive to Azoxymethane (AOM)/Dextran sodium sulfate (DSS) induced Colitis-Associated Cancer (Ahn et al., Oncogene, 34:5302-5308, 2015) STING resides in the endoplasmic reticulum (ER) of hematopoietic cells as well as endothelial and epithelial cells and controls the induction of numerous host defense genes, such as type I IFN as well as pro-inflammatory genes including IL1-β in response to the detection of cyclic dinucleotides (CDNs) such as cyclic-di-AMP (c-di-AMP) generated from intracellular bacteria (Ishikawa and Barber, Nature 455:674-678, 2008; Woodward et al., Science 328:1703-1705, 2010). STING is also the sensor for CDNs produced from a cellular nucleotidyltransferase referred to as cGAS (cyclic GMP-AMP synthase, also referred to as Mab-21 Domain-Containing Protein and C6orf150) (Sun et al., Science 339:786-791, 2013). Cytosolic DNA species which can constitute the genome of invading pathogens such as HSV-1, or plausibly self-DNA leaked from the nucleus can bind to cGAS to generate non-canonical cGAMP containing one 2′-5′ phosphodiester linkage and a canonical 3-5′ linkage (c[G(2′,5′)pA(3′,5′)p]). The STING pathway may recognize damaged DNA during early response to intestinal damage and may be essential for invigorating tissue repair pathways involving IL1 β and IL-18 (Ahn et al., 2015, supra). STING has also been recently reported to play an essential role in dendritic cell recognition of dying tumor cells and the priming of anti-tumor cytotoxic T-cell (CTL) responses (Corrales et al., Cell reports 11:1018-1030, 2015; Woo et al., Immunity 41:830-842, 2014). Thus, while loss of STING may facilitate tumorigenesis through preventing wound repair and by preventing the production of tumor specific CTLs, the effectiveness of STING signaling in human tumors remains unknown.

### SUMMARY

In one aspect, described herein is a vector comprising a human STimulator of INterferon Genes (STING) polynucleotide encoding a STING protein and a cyclic GMP-AMP synthase (cGAS) polynucleotide encoding a cGAS protein. In some embodiments, the STING protein is a constitutively active STING protein. In some embodiments, the constitutively active STING comprises from a mutation at amino acid 284 of SEQ ID NO:1, optionally wherein the mutation is R284S of SEQ ID NO:1.

In some embodiments, the cGAS protein is a constitutively active cGAS protein. In some embodiments, the constitutively active cGAS protein is DCNV.

In some embodiments, the vector comprises a polynucleotide encoding a constitutively active STING protein and a constitutively active cGAS protein. In some embodiments, the vector comprises a polynucleotide encoding a STING protein and a constitutively active cGAS protein. In some embodiments, the vector comprises a polynucleotide encoding a constitutively active STING protein and a cGAS protein.

In any of the embodiments described herein, the vector is a viral or plasmid vector. In some embodiments, the viral vector is selected from the group consisting of vesicular stomatitis virus (VSV), a herpes simplex virus (HSV), a lentivirus, an adenovirus, an adeno-associated virus, a vaccinia virus and a modified vaccinia Ankara (MVA) virus. In some embodiments, the vector is a VSV vector. In some embodiments, the vector is an HSV-1 vector.

Compositions comprising the vectors described herein are also contemplated. In various embodiments, the composition is a pharmaceutical composition comprising a pharmaceutically acceptable excipient.

In some embodiments, the composition is administered intratumorally, intravenously, intra-arterially, intraperitoneally, intranasally, intramuscularly, intradermally or subcutaneously. In some embodiments, the composition induces infiltration of immune cells into the tumor. In some embodiments, the immune cells are macrophages or other phagocytes.

In another aspect, described herein is a method of stimulating an immune response in a subject in need thereof comprising administering a composition comprising a vector (or vaccine) described herein to the subject, wherein the composition induces STING signaling. In some embodiments, the subject is suffering from cancer.

In another aspect, described herein is a method of treating cancer in a subject comprising administering a composition comprising a vector (or vaccine) described herein to the subject, wherein the composition induces STING signaling. In some embodiments, the cancer is ovarian cancer, colon cancer, melanoma, breast cancer or lung cancer. In some embodiments, tumor size in the subject is decreased by about 25-50%, about 40-70% or about 50-90% or more.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

Other features and advantages of the present disclosure will become apparent from the following detailed description. It should be understood, however, that the detailed description and the specific examples, while indicating preferred embodiments of the disclosure, are given by way of illustration only, because various changes and modifications within the spirit and scope of the disclosure will become apparent to those skilled in the art from this detailed description.

### BRIEF DESCRIPTION OF THE FIGURES

FIGS. 1A-1D demonstrate the oncolytic activity of VSV-DncV and VSV-R284S STING. FIG. 1A shows the generation of rVSV expressing R283S STING or DncV. FIG. 1B shows that the rVSV expresses R283S STING or DncV. FIG. 1C shows that rVSV-DncV generates 3′3′ cGAMP in 293T cells. FIG. 1D shows that rVSV-DncV or rVSV-R283S STING enhance anti-tumor activity in B16 melanoma.

FIGS. 2A-2D demonstrate the oncolytic activity of HSV-DncV and HSV-STING. FIG. 2A shows the genetic structure and expression of rHSV1. FIG. 2B shows the reconstitution of STING/cGAS in 293T cells. FIG. 2C shows the rescue of STING/cGAS pathway in colon cancer cells. FIG. 2D is a graph showing that rHSV1 therapy reduced tumor volume in B16 melanoma cells.

FIGS. 3A-3F show that rHSV1 therapy results in an antigen specific memory response. FIG. 3A is a graph showing that rHSV1 therapy reduced tumor volume in B16 melanoma cells. FIG. 3B is a bar graph showing that OVA antigen specific-IFNγ production in CD8+T cells in the spleen from tumor bearing C57/BL6 mice injected with rHSV1 vectors. FIG. 3C is a bar graph showing the level of HSV specific-IFNγ production in CD8+ T cells from the spleens of tumor bearing C57/BL6 mice injected with rHSV1 vectors. FIG. 3D is a bar graph showing the levels of CD4+ and CD8+ cells in the activated T cell population (CD44^{hl}) from the spleens of tumor bearing C57/BL6 mice injected with rHSV1 vectors. FIG. 3E is a bar graph showing the levels of CD4+ and CD8+ T cell populations from the spleens of tumor bearing C57/BL6 mice injected with rHSV1 vectors. FIG. 3F is a bar graph showing the levels of OVE specific CD8+ T cells in the spleens of tumor bearing C57/BL6 mice injected with rHSV1 vectors.

### DETAILED DESCRIPTION

Unless otherwise defined, all technical and scientific terms used herein have the same meaning as commonly understood by one of ordinary skill in the art to which this invention belongs. The following references provide one of skill with a general definition of many of the terms used in this invention: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

It is noted here that, as used in this specification and the appended claims, the singular forms “a,” “an,” and “the” include plural reference unless the context clearly dictates otherwise.

As used herein, the following terms have the meanings ascribed to them unless specified otherwise.

### Definitions

The term “STimulator of INterferon Genes” or “STING” as used herein includes, without limitation, nucleic acids, polynucleotides, oligonucleotides, sense and antisense polynucleotide strands, complementary sequences, peptides, polypeptides, proteins, homologous and/or orthologous STING molecules, isoforms, precursors, mutants, variants, derivatives, splice variants, alleles, different species, and active fragments thereof.

An “constitutively active STING protein” refers to mutant of STING protein which results in a gain-of-function mutant in which STING is constitutively active. An active STING mutant is also a STING variant polynucleotide having a mutation in the wild type STING protein. Exemplary constitutively active mutations include, but are not limited to, N154S or R284S of SEQ ID NO:1.

The term “cyclic GMP-AMP synthase” or “cGAS” as used herein includes, without limitation, nucleic acids, polynucleotides, oligonucleotides, sense and antisense polynucleotide strands, complementary sequences, peptides, polypeptides, proteins, homologous and/or orthologous cGAS molecules, isoforms, precursors, mutants, variants, derivatives, splice variants, alleles, different species, and active fragments thereof.

A “vector” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest in gene therapy. Vectors include, for example, viral vectors, such as vesicular stomatitis virus (VSV), lentivirus, adenovirus, adeno-associated virus, vaccinia virus, herpes simplex virus, or modified vaccinia Ankara (MVA) virus vectors, liposomes and other lipid-containing complexes, and other macromolecular complexes capable of mediating delivery of a polynucleotide to a host cell. Vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector. It is contemplated that the vectors can comprises a polynucleotide encoding a STING protein or constitutively active STING and a polynucleotide encoding a cGAS protein or constitutively active cGAS, as well as a polynucleotide encoding another protein that may improve efficacy of the vector.

Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34:167-171 (2003). A large variety of such vectors are known in the art and are generally available.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

A “DNA Vaccine” or “DNA vector” as used herein refers to a synthetic DNA structure that can be transcribed in target cells and can comprise a linear nucleic acid such as a purified DNA, a DNA incorporated in a plasmid vector, or a DNA incorporated into any other vector suitable for introducing DNA into a host cell. In various embodiments, the DNA vaccine can be naked DNA. Provided herein is a naked DNA vaccine, a plasmid DNA vaccine or a viral vector vaccine. It is contemplated that the vaccine is a live viral vaccine, live attenuated viral vaccine, or inactivated or killed viral vaccine. In various embodiments, the vaccine may comprise virus-like particles (VLPs).

“Vesicular stomatitis virus” or “VSV” as used herein refers to any strain of VSV or mutant forms of VSV, such as those described in WO 01/19380 or US20140088177. A VSV construct herein may be in any of several forms, including, but not limited to, genomic RNA, mRNA, cDNA, part or all of the VSV RNA encapsulated in the nucleocapsid core, VSV complexed with compounds such as PEG and VSV conjugated to a nonviral protein. VSV vectors useful herein encompass replication-competent and replication-defective VSV vectors, such as, VSV vectors lacking G glycoprotein.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, genomic RNA, mRNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P-NH2) or a mixed phosphoramidate-phosphodiester oligomer. Peyrottes et al. (1996) Nucleic Acids Res. 24:1841-8; Chaturvedi et al. (1996) Nucleic Acids Res. 24:2318-23; Schultz et al. (1996) Nucleic Acids Res. 24:2966-73. A phosphorothioate linkage can be used in place of a phosphodiester linkage. Braun et al. (1988) J. Immunol. 141:2084-9; Latimer et al. (1995) Molec. Immunol. 32:1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. Reference to a polynucleotide sequence (such as referring to a SEQ ID NO) also includes the complement sequence.

The following are non-limiting examples of polynucleotides: a gene or gene fragment, exons, introns, genomic RNA, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracil, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

The phrase “substantially homologous” or “substantially identical” in the context of two nucleic acids or polypeptides, generally refers to two or more sequences or subsequences that have at least 40%, 60%, 80%, 90%, 95%, 96%, 97%, 98% or 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of either or both comparison biopolymers. It is contemplated herein that the STING and/or cGAS protein useful in the VSV vector and immunogenic composition, vaccine or viral particle can have 80%, 90%, 95%, 96%, 97%, 98% or 99% nucleotide or amino acid residue identity to a naturally-occurring STING and/or cGAS protein.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Natl. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, WI), or by visual inspection. Alignment is also measured using such algorithms as PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Another algorithm that is useful for generating multiple alignments of sequences is Clustal W (Thompson et al., Nucleic Acids Research 22:4673-4680, 1994). Another example of algorithm that is suitable for determining percent sequence identity and sequence similarity is the BLAST algorithm, which is described in Altschul et al., J. Mol. Biol. 215:403-410, 1990. Software for performing BLAST analyses is publicly available through the National Center for Biotechnology Information.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Under transcriptional control” is a term well understood in the art and indicates that transcription of a polynucleotide sequence depends on its being operably (operatively) linked to an element which contributes to the initiation of, or promotes, transcription. “Operably linked” refers to a juxtaposition wherein the elements are in an arrangement allowing them to function.

As used herein, in the context of the viral vectors, a “heterologous polynucleotide” or “heterologous gene” or “transgene” is any polynucleotide or gene that is not present in wild-type viral vector.

As used herein, in the context of the viral vectors, a “heterologous” promoter is one which is not associated with or derived from the viral vector itself.

A “host cell” includes an individual cell or cell culture which can be or has been a recipient of a VSV vector(s) described herein. Host cells include progeny of a single host cell, and the progeny may not necessarily be completely identical (in morphology or in total DNA complement) to the original parent cell due to natural, accidental, or deliberate mutation and/or change. A host cell includes cells transfected, transformed or infected in vivo or in vitro with a vector herein.

“Replication” and “propagation” are used interchangeably and refer to the ability of a vector of the invention to reproduce or proliferate. These terms are well understood in the art. For purposes of this disclosure, replication involves production of viral proteins and is generally directed to reproduction of the viral vector. Replication can be measured using assays standard in the art. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

As used herein, “vaccine” refers to a composition comprising a vector comprising a polynucleotide encoding a STING protein and a polynucleotide encoding a cGAS protein as described herein, which is useful in the treatment of cancer or other conditions in which enhanced immune response is indicated. It is contemplated that the vaccine comprises a pharmaceutically acceptable carrier and/or an adjuvant. It is contemplated that vaccines are prophylactic or therapeutic. A “prophylactic” treatment is a treatment administered to a subject who does not exhibit signs of a disease or exhibits only early signs for the purpose of decreasing the risk of developing pathology. The compounds of the invention may be given as a prophylactic treatment to reduce the likelihood of developing a pathology or to minimize the severity of the pathology, if developed. A “therapeutic” treatment is a treatment administered to a subject who exhibits signs or symptoms of pathology for the purpose of diminishing or eliminating those signs or symptoms. The signs or symptoms may be biochemical, cellular, histological, functional, subjective or objective.

The term “induces or enhances an immune response” as used herein refers to causing a statistically measurable induction or increase in an immune response over a control sample to which the peptide, polypeptide or protein has not been administered. Preferably the induction or enhancement of the immune response results in a prophylactic or therapeutic response in a subject. Examples of immune responses are increased production of type I IFN, increased resistance to viral and other types of infection by alternate pathogens. The enhancement of immune responses to tumors (anti-tumor responses), or the development of vaccines to prevent tumors or eliminate existing tumors.

The “treatment of cancer”, as that phrase is used herein refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

As used herein, “isolated” refers to a virus or immunogenic composition that is removed from its native environment. Thus, an isolated biological material is free of some or all cellular components, i.e., components of the cells in which the native material occurs naturally (e.g., cytoplasmic or membrane component). In one aspect, a virus or antigenic composition is deemed isolated if it is present in a cell extract or supernatant. In the case of nucleic acid molecules, an isolated nucleic acid includes a PCR product, an isolated mRNA, a cDNA, or a restriction fragment.

“Purified” as used herein refers to a virus or immunogenic composition that has been isolated under conditions that reduce or eliminate the presence of unrelated materials, i.e., contaminants, including endogenous materials from which the composition is obtained. By way of example, and without limitation, a purified virion is substantially free of host cell or culture components, including tissue culture or cell proteins and non-specific pathogens. In various embodiments, purified material substantially free of contaminants is at least 50% pure; at least 60%, at least 70%, at least 80%, at least 90%, at least 95%, at least 96%, at least 97%, at least 98% or even at least 99% pure. Purity can be evaluated by chromatography, gel electrophoresis, immunoassay, composition analysis, biological assay, and other methods known in the art.

As used herein, “pharmaceutical composition” refers to a composition suitable for administration to a subject animal, including humans and mammals. A pharmaceutical composition comprises a pharmacologically effective amount of a virus or antigenic composition of the invention and also comprises a pharmaceutically acceptable carrier. A pharmaceutical composition encompasses a composition comprising the active ingredient(s), and the inert ingredient(s) that make up the pharmaceutically acceptable carrier, as well as any product which results, directly or indirectly, from combination, complexation or aggregation of any two or more of the ingredients. Accordingly, the pharmaceutical compositions of the present invention encompass any composition made by admixing a compound or conjugate of the present invention and a pharmaceutically acceptable carrier.

As used herein, “pharmaceutically acceptable carrier” include any and all clinically useful solvents, dispersion media, coatings, antibacterial and antifungal agents, isotonic and absorption delaying agents, buffers, and excipients, such as a phosphate buffered saline solution, 5% aqueous solution of dextrose or mannitol, and emulsions, such as an oil/water or water/oil emulsion, and various types of wetting agents and/or adjuvants. Suitable pharmaceutical carriers and formulations are described in Remington's Pharmaceutical Sciences, 19th Ed. (Mack Publishing Co., Easton, 1995). Pharmaceutical carriers useful for the composition depend upon the intended mode of administration of the active agent. Typical modes of administration include, but are not limited to, enteral (e.g., oral) or parenteral (e.g., subcutaneous, intramuscular, intravenous or intraperitoneal injection; or topical, transdermal, or transmucosal administration). A “pharmaceutically acceptable salt” is a salt that can be formulated into a compound or conjugate for pharmaceutical use including, e.g., metal salts (sodium, potassium, magnesium, calcium, etc.) and salts of ammonia or organic amines.

As used herein, “pharmaceutically acceptable” or “pharmacologically acceptable” refers to a material which is not biologically or otherwise undesirable, i.e., the material may be administered to an individual without causing any undesirable biological effects or interacting in a deleterious manner with any of the components of the composition in which it is contained, or when administered using routes well-known in the art, as described below.

### STING (Stimulator of Interferon Genes) Stimulating Pathway

STING (Stimulator of Interferon Genes), a molecule that plays a key role in the innate immune response, includes 5 putative transmembrane (TM) regions, predominantly resides in the endoplasmic reticulum (ER), and is able to activate both NF-κB and Interferon Regulatory Factor 3 (IRF3) transcription pathways to induce type I IFN and to exert a potent anti-viral state following expression. Human STING is a 379 amino acid protein, having an amino acid sequence set out in Genbank Accession No. NP_938023 and nucleotide sequence set out in Genbank Accession No. NM_198282, though alternate protein isoforms may exist (Genbank Accession Nos. NP_001288667.1, XP_011535942.1, XP_011535941.1). See e.g., U.S. patent publication 20130039933 and PCT/US2009/052767, herein incorporated by reference in their entirety. The amino acid sequence of human STING (379 amino acids) (SEQ ID NO:1) is set out below:

MQPWHGKAMQ RASEAGATAP KASARNARGA PMDPTESPAA
PEAALPKAGK FGPARKSGSR QKKSAPDTQE RPPVRATGAR
AKKAPQRAQD TQPSDATSAP GAEGLEPPAA REPALSRAGS
CRQRGARCST KPRPPPGPWD VPSPGLPVSA PILVRRDAAP
GASKLRAVLE KLKLSRDDIS TAAGMVKGVV DHLLLRLKCD
SAFRGVGLLN TGSYYEHVKI SAPNEFDVMF KLEVPRIQLE
EYSNTRAYYF VKFKRNPKEN PLSQFLEGEI LSASKMLSKF
RKIIKEEIND IKDTDVIMKR KRGGSPAVTL LISEKISVDI
TLALESKSSW PASTQEGLRI QNWLSAKVRK QLRLKPFYLV
PKHAKEGNGF QEETWRLSFS HIEKEILNNH GKSKTCCENK
EEKCCRKDCL KLMKYLLEQL KERFKDKKHL DKFSSYHVKT
AFFHVCTQNP QDSQWDRKDL GLCFDNCVTY FLQCLRTEKL
ENYFIPEFNL FSSNLIDKRS KEFLTKQIEY ERNNEFPVFD EF
MPHSSLHPSIPCPRGHGAQKAALVLLSACLVTLWGLGEPPEHTLRY
LVLHLASLQLGLLLNGVCSLAEELRHIHSRYRGSYWRTVRACLGCP
LRRGALLLLSIYFYYSLPNAVGPPFTWMLALLGLSQALNILLGLKG
LAPAEISAVCEKGNFNVAHGLAWSYYIGYLRLILPELQARIRTYNQ
HYNNLLRGAVSQRLYILLPLDCGVPDNLSMADPNIRFLDKLPQQTG
DRAGIKDRVYSNSIYELLENGQRAGTCVLEYATPLQTLFAMSQYSQ
AGFSREDRLEQAKLFCRTLEDILADAPESQNNCRLIAYQEPADDSS
FSLSQEVLRHLRQEEKEEVTVGSLKTSAVPSTSTMSQEPELLISGM
EKPLPLRTDFS

Loss of STING reduced the ability of polyI:C to activate type I IFN and rendered murine embryonic fibroblasts lacking STING (^{-/-}MEFs) generated by targeted homologous recombination, susceptible to vesicular stomatitis virus (VSV) infection. In the absence of STING, DNA-mediated type I IFN responses were inhibited, indicating that STING may play an important role in recognizing DNA from viruses, bacteria, and other pathogens which can infect cells. Yeast-two hybrid and co-immunoprecipitation studies indicated that STING interacts with RIG-I and with Ssr2/TRAPβ, a member of the translocon-associated protein (TRAP) complex required for protein translocation across the ER membrane following translation. RNAi ablation of TRAPβ inhibited STING function and impeded the production of type I IFN in response to polyIC (Ishikawa and Barber, Nature 455:674-678, 2008).

Additional experiments have shown that STING itself binds nucleic acids including single- and double-stranded DNA such as from pathogens and apoptotic DNA, and plays a central role in regulating proinflammatory gene expression in inflammatory conditions such as DNA-mediated arthritis and cancer. Certain inhibitors and activators of STING are discussed in International Patent Publication No. WO 2013/166000.

### Cyclic GMP-AMP Synthase (cGAS)

Cytosolic DNA (CDN) species trigger STING signalling following binding to a protein termed cyclic GMP-AMP synthase (cGAS). Human cGAS is a 522 amino acid protein, having an amino acid sequence set out in Genbank Accession No. NP_612450 and nucleotide sequence set out in Genbank Accession No. NM_138441.2, though alternate protein isoforms may exist (Genbank Accession Nos. XP_016865721.1). The amino acid sequence of human cGAS (SEQ ID NO:2) is as follows:

MQPWHGKAMQ RASEAGATAP KASARNARGA PM [symbol] PTESPAA
PEAA [symbol] PKAGK FGPARKSGSR QKKSAPDTQE R [symbol] PVRA [symbol] [symbol] AR
AKKAPQRAQD [symbol] QPSDA [symbol] SAP [symbol] AE [symbol] [symbol] EPPAA REPA [symbol] SRAGS
CRQRGARCST KPRPPPGPWD VPSPGLPVSA PILVRRDAAP
GASK [symbol] RAVLE K [symbol] KLSRDD [symbol] S TAAGMVKGVV DHLL [symbol] R [symbol] KCD
SAFRGVGLLN TGSYYEHVKI SAPNEFDVMF KLEVPR [symbol] QLE
EYSNTRAYYF VKFKRNPKEN P [symbol] SQFLEGE [symbol] [symbol] SASKM [symbol] SKF
RK [symbol] [symbol] KEE [symbol] ND [symbol] KDTDV [symbol] MKR KRGGSPAVTL LISEK [symbol] SVDI [symbol] [symbol]
A [symbol] ESKSSW PASTQEG [symbol] RI QNWLSAKVRK Q [symbol] RLKPFYLV
PKHAKEGNGF QEE [symbol] WR [symbol] SFS H [symbol] EKE [symbol] LNNH GKSKTCCENK
EEKCCRKDCL KLMKY [symbol] [symbol] EQL KERFKDKKHL DKFSSYHVKT
AFFHVCTQNP QDSQWDRKD [symbol] GLCFDNCVTY F [symbol] QCLRTEKL
ENYF [symbol] PEFNL FSSNLIDKRS KEFLTKQIEY ERNNEFPVFD EF [symbol]
indicates data missing or illegible when filed

In the presence of ATP and GTP, cGAS catalyzes the production of a type of CDN referred to as cGAMP (cyclic GMP-AMP), which contains one 2′,5′-phosphodiester linkage and a canonical 3′,5′ linkage (c[G(2′,5′)pA(3′,5′)p]) (Sun et al., Science, 339:786-791, 2013; Diner et al., Cell Re., 3:1355-1361, 2013 and Ablasser et al., Nature, 498:380-384, 2013). STING is also known to bind double-stranded DNA (dsDNA) directly (Abe et al., Mol. Cell., 50:5-15, 2013), although the physiological relevance of this remains to be clarified.

cGAS is a member of the nucleotidyltransferase family that includes the human dsRNA sensor oligoadenylate synthetase 1 (OAS1). Sequences of non-specific dsDNA species greater than 30 bp have been reported to stimulate cGAS activity, with a single CDN generated by cGAS binding to two molecules of STING in the ER (Suzuki, FEBS Lett. 584:1280-6, 2010). This event probably influences changes in STING conformation, which leads to a striking trafficking event in which STING, complexed with TANK-binding kinase 1 (TBK1), relocates to perinuclear regions of the cell (Barber, Trends Immunol., 35:88-93, 2014; Kohno et al., Cell. 2013;155:688-98, 2013. This process is required to deliver TBK1 to endolysosomal compartments where it phosphorylates the transcription factors interferon regulatory factor 3 (IRF3) and nuclear factor-κB (NF-κB). These transcription factors then translocate into the nucleus to initiate innate immune gene transcription. STING is then rapidly degraded, an event that may avoid problems associated with sustained cytokine production (Konno et al., Cell, 155:688-698, 2013).

### DncV

In contrast to mammalian cGAS, DncV is a constitutively active enzyme and can produce three different types of CDNs (i.e., 3′-5′ cGAMP, c-di-GMP, and c-di-AMP) in the absence of bound DNA (Davies et al., Cell, 149:358-370, 2012; Diner et al., 3:1355-1361, 2013; Kato et al., Structure 23:843-850, 2015). However, when GTP and ATP are used as substrates in vitro, the predominant product of DncV is 3′-5′ cGAMP. Similarly, the major product of DncV in vivo, where both GTP and ATP are present, was also shown to be 3′-5′ cGAMP (Davies et al., 2012; Diner et al., 2013). Quite recently, the crystal structures of V. cholerae DncV (VcDncV) were reported from two groups independently. Zhu et al. (Mol. Cell, 55:931-937, 2014) determined the structure of VcDncV with 5-methyltetrahydrofolate diglutamate, a folate analog, and revealed the mechanism of the catalytic activity regulation by folates. Kranzusch et al. (Cell, 158:1011-1021, 2014) determined the structures of VcDncV in the apo GTP analog (guanosine-5′-(α,β)-methyleno triphosphate, GMPCPP)-bound and pppA(3-5′)pG-bound forms. The GMPCPP-bound form is considered to be a state before the first reaction step (pre-reaction state), while the pppA(3′-5′)pG-bound form is regarded as a state before the second reaction step (intermediate state). Analyses of these structures suggested that DncV produces 3′-5′ cGAMP in a similar manner to cGAS, except that its acceptor and donor pockets bind to ATP and GTP, respectively, to form pppA(3′-5′)pG as the product of the first reaction step. Moreover, based on a structural comparison between VcDncV and mouse cGAS in the intermediate state, it was proposed that Ile376 (Arg376 in human cGAS) is critical for their distinct linkage specificities, i.e., 2′-5′ versus 3′-5′ (Kranzusch et al., 2014). However, the substrate recognition mechanism in the pre-reaction state, which defines the 3′-5′ phosphodiester linkage specificity, still remains elusive because the electron density for the acceptor nucleotide was not observed in the pre-reaction state.

### Vesicular Stomatitis Virus (VSV)

Vesicular stomatitis virus (VSV) is a nonsegmented, negative-strand RNA virus that belongs to the family of rhabdoviridae (Barber, G., Oncogene. 24(52):7710-9, 2005) widely used as a vaccine platform as well as an anticancer therapeutic. VSV comprises approximately an 11 kilobase genome that encodes for five proteins referred to as the nucleocapsid (N), polymerase proteins large (L) and (P) (formerly termed NS, originally indicating nonstructural), surface glycoprotein (G) and a peripheral matrix protein (M). The virus particles contain a helical, nucleocapsid core composed of the genomic RNA and protein. The genome is tightly encased in nucleocapsid protein and also comprises the polymerase proteins L and P. An additional matrix (M) protein lies within the membrane envelope, perhaps interacting both with the membrane and the nucleocapsid core. A single glycoprotein (G) species spans the membrane and forms the spikes on the surface of the virus particle. Glycoprotein G is responsible for binding to cells and membrane fusion.

The VSV genome is the negative sense (i.e., complementary to the RNA sequence (positive sense) that functions as mRNA to directly produce encoded protein), and rhabdoviruses must encode and package an RNA-dependent RNA polymerase in the virion (Baltimore et al., 1970, Proc. Natl. Acad. Sci. USA 66:572-576), composed of the P and L proteins. This enzyme transcribes genomic RNA to make subgenomic mRNAs encoding the 5-6 viral proteins and also replicates full-length positive and negative sense RNAs. The genes are transcribed sequentially, starting at the 3′ end of the genomes.

The sequences of the VSV mRNAs and genome is described in Gallione et al. 1981, J. Virol. 39:529-535; Rose and Gallione, 1981, J. Virol. 39:519-528; Rose and Schubert, 1987, Rhabdovirus genomes and their products, p. 129-166, in R. R. Wagner (ed.), The Rhabdoviruses. Plenum Publishing Corp., NY; Schubert et al., 1985, Proc. Natl. Acad. Sci. USA 82:7984-7988. WO 96/34625 published Nov. 7, 1996, disclose methods for the production and recovery of replicable vesiculovirus. U.S. Pat. No. 6,168,943, issued Jan. 2, 2001, describes methods for making recombinant vesiculoviruses (e.g., VSV).

VSV is predominantly a pathogen of livestock (Letchworth et al., Vet. J. 157:239-260, 1999) and usually produces a self-limiting disease in livestock. It is essentially non-pathogenic in humans (Balachandran and Barber (2000, IUBMB Life 50:135-8), but does, however, have a very broad species tropism. The cellular tropism of VSV is determined predominantly at postentry steps, since the G glycoprotein of the virus mediates entry into most tissues in nearly all animal species (Carneiro et al., J. Virol. 76:3756-3764, 2002). Though viral entry can take place in nearly all cell types (Kelly et al., J Virol. 84(3):1550-1562, 2010), in vivo models of VSV infection have revealed that the virus is highly sensitive to the innate immune response, limiting its pathogenesis (Barber, G. N. Oncogene 24:7710-7719, 2005). VSV is intensively responsive to type I interferon (IFN), as the double-stranded RNA (dsRNA)-dependent PKR (Balachandran, S., and G. N. Barber. Cancer Cell 5:51-65, 2004), the downstream effector of pattern recognition receptors MyD88 (Lang et al., Eur. J. Immunol. 37:2434-2440, 2007), and other molecules mediate shutdown of viral translation and allow the adaptive immune response to clear the virus.

VSV induces potent in vitro and in vivo tumor cytotoxic effects, and its efficacy has been tested in a number of xenograft and syngeneic models. VSV-induced neurotoxicity, however is dose limiting (Clarke et al., Springer Semin Immunopathol. 2006; 28(3):239-53, 2006; Johnson et al., Virology. 2007; 360(I):36-49), and can limit clinical development efforts of this agent (Kurisetty et al., Head Neck. 36(11):1619-1627, 2014).

A table of various VSV strains is shown in “Fundamental Virology”, second edition, supra, at page 490. WO 01/19380 and U.S. Pat. No. 6,168,943 disclose that strains of VSV include Indiana, New Jersey, Piry, Colorado, Coccal, Chandipura and San Juan. The complete nucleotide and deduced protein sequence of a VSV genome is known and is available as Genbank VSVCG, accession number JO2428; NCBI Seq ID 335873; and is published in Rose and Schubert, 1987, in The Viruses: The Rhabdoviruses, Plenum Press, NY. pp. 129-166. A complete sequence of a VSV strain is shown in U.S. Pat. No. 6, 168,943. VSV New Jersey strain is available from the American Type Culture Collection (ATCC) and has ATCC accession number VR-159. VSV Indiana strain is available from the ATCC and has ATCC accession number VR-1421.

The present disclosure provides recombinant vesicular stomatitis virus (VSV) vectors comprising nucleic acid encoding a STING protein and a cGAS polynucleotide encoding a cGAS protein. The present disclosure contemplates VSV vectors comprising nucleic acid encoding more than one biologically active protein, such as for example, a VSV vector comprising a nucleic acid encoding a STING protein and a nucleic acid encoding a cGAS protein. In some embodiments, the STING protein is a constitutively active STING protein. In some embodiments, the cGAS protein is a constitutively active STING protein (DCNV).

In other examples, the VSV vector is replication-competent. In additional examples, the VSV vector is replication-defective. In yet other examples, the VSV vector lacks a protein function essential for replication, such as G-protein function or M and/or N protein function. The VSV vector may lack several protein functions essential for replication. In further embodiments, the subject or patient is an animal, preferably a mammal, such as a human. The present disclosure also provides viral particles comprising a VSV vector, such as a VSV vector comprising nucleic acid encoding a STING protein and a nucleic acid encoding a cGAS protein. The present disclosure also contemplates isolated nucleic acid encoding a recombinant VSV vector herein as well as host cells comprising a recombinant VSV vector of described herein.

In various embodiments, the VSV vector further comprises one or more deletions or mutations in one or more VSV nucleic acid sequences. A wild-type VSV genome has the following gene order: 3′-NPMGL-5′. In one embodiment, the VSV vector may lack a G protein sequence or it may have one or more mutations which result in a VSV vector lacking G-protein function or express a mutated or truncated G-protein. In another embodiment, the VSV vector has mutations or deletions of M sequences, producing VSV vectors which do not express M protein or lack M protein function or express a mutated or truncated M protein. In one embodiment, a VSV vector of the disclosure comprises one or more mutations in its genome. For example, a vector of the disclosure includes, but is not limited to, a VSV temperature-sensitive N gene mutation, a temperature-sensitive L gene mutation, a point mutation, a G-stem mutation, a non-cytopathic M gene mutation, a gene shuffling or rearrangement mutation, a truncated G gene mutation, an antisense RNA mutation, a G gene insertion mutation, a gene deletion mutation and the like. Thus the term, a “mutation” includes mutations known in the art as insertions, deletions, substitutions, gene rearrangement or shuffling modifications.

In various embodiments, for the VSV vectors described herein, a polynucleotide sequence may also encode one or more heterologous (or foreign) polynucleotide sequences or open reading frames (ORFs). The foreign polynucleotide sequences can vary as desired, and include, but are not limited to STING proteins, cGAS proteins, or other protein of interest. In preferred embodiments, a foreign nucleic acid can be inserted into regions of VSV encoding for G-protein, M-protein or combinations thereof.

In other embodiments, a composition comprises an attenuated vesicular stomatitis (VSV) vector or an attenuated Herpes Simplex Virus (HSV) vector expressing a one or more oligonucleotides which modulate expression or function of target molecules. In various embodiments, the oligonucleotides comprises: dsRNA, siRNA, antisense RNA, RNA, enzymatic RNA or microRNA.

Also contemplated herein is an immunogenic composition comprising a vesicular stomatitis virus (VSV) vector or Herpes Simplex Virus (HSV) vector comprising a nucleic acid encoding a STING protein and a nucleic acid encoding a cGAS protein described herein.

The disclosure also provides a vaccine comprising a vesicular stomatitis virus (VSV) vector or Herpes Simplex Virus (HSV) vector comprising a nucleic acid encoding a STING protein, a nucleic acid encoding a cGAS protein, and an adjuvant.

### HSV Vectors

Herpes Simplex Virus (HSV) 1 and 2 are members of the Herpesviridae family, which infect humans. The HSV genome contains two unique regions, which are designated unique long (UL) and unique short (US) region. Each of these regions is flanked by a pair of inverted terminal repeat sequences. There are about 75 known open reading frames. The viral genome has been engineered to develop oncolytic viruses for use in e.g. cancer therapy. Tumor-selective replication of HSV is conferred by mutation of the HSV ICP34.5 (also called y34.5) gene. HSV contains two copies of ICP34.5. Mutants inactivating one or both copies of the ICP34.5 gene are known to lack neurovirulence, i.e. be avirulent/non-neurovirulent and be oncolytic.

Suitable HSV for use according to the disclosure may be derived from either HSV-1 or HSV-2, including any laboratory strain or clinical isolate. In some embodiments, the oHSV may be or may be derived from one of laboratory strains HSV-1 strain 17, HSV-1 strain F, or HSV-2 strain HG52. In other embodiments, it may be of, or derived from, non-laboratory strain JS-1. Other suitable HSV-1 viruses include, but are not limited to HrrR3, G2O7, G47Delta, HSV 1716, HF10, NV1020, T-VEC, J100, M002, NV1042, G1O7-IL2, rQNestin34.5 and G47Δ-mIL-18.

In some embodiments, the HSV has one or both of the y34.5 genes modified such that it is incapable of expressing a functional ICP34.5 protein. The genes may be modified by mutation of one or more nucleotides, insertions, deletions, substitutions, etc. The alteration may be in the coding sequence, non-coding sequence (e.g., promoter) or both. In some embodiments, both copies of the γ34.5 genes are mutated.

The HSV may have additional mutations, which may include disabling mutations e.g., deletions, substitutions, insertions), which may affect the virulence of the virus or its ability to replicate. For example, mutations may be made in any one or more of ICP6, ICPO, ICP4, ICP27, ICP47, ICP 24, ICP56. Preferably, a mutation in one of these genes (optionally in both copies of the gene where appropriate) leads to an inability (or reduction of the ability) of the HSV to express the corresponding functional polypeptide. In some embodiments, the promoter of a viral gene may be substituted with a promoter that is selectively active in target cells or inducible.

The present disclosure provides recombinant Herpes Simplex Virus (HSV) vectors comprising a nucleic acid encoding a STING protein and a nucleic acid encoding a cGAS protein. The present disclosure contemplates HSV vectors comprising nucleic acid encoding more than one biologically active protein, such as for example, a HSV vector comprising a nucleic acid encoding a STING protein and a nucleic acid encoding a cGAS protein. In some embodiments, the STING protein is a constitutively active STING protein. In some embodiments, the cGAS protein is a constitutively active cGAS protein (DCNV).

In other examples, the HSV vector is replication-competent. In additional examples, the HSV vector is replication-defective. In yet other examples, the HSV vector lacks a protein function essential for replication. The HSV vector may lack several protein functions essential for replication. In further embodiments, the subject or patient is an animal, preferably a mammal, such as a human. The present disclosure also provides viral particles comprising a HSV vector, such as a HSV vector comprising nucleic acid encoding a STING protein and a nucleic acid encoding a cGAS protein. The present disclosure also contemplates isolated nucleic acid encoding a recombinant HSV vector herein as well as host cells comprising a recombinant HSV vector described herein.

In various embodiments, for the HSV vectors described herein, a polynucleotide sequence may also encode one or more heterologous (or foreign) polynucleotide sequences or open reading frames (ORFs). The foreign polynucleotide sequences can vary as desired, and include, but are not limited to STING proteins, cGAS proteins, or other protein of interest.

### Other Viral Vectors

It is further contemplated that the viral vector for the vaccine is a retrovirus, including a lentivirus, an adenovirus, an adeno-associated virus, a vaccinia virus, or a modified vaccinia Ankara (MVA) virus. Constructs as described above with respect to the STING protein and/or cGAS protein can also be made in these other viral vectors. For example, the present disclosure provides recombinant vectors comprising nucleic acid encoding a STING protein and/or a cGAS protein, wherein said recombinant vector expresses the STING protein and cGAS protein and is useful for cancer therapy.

Retroviruses are enveloped RNA viruses that are capable of infecting animal cells, and that utilize the enzyme reverse transcriptase in the early stages of infection to generate a DNA copy from their RNA genome, which is then typically integrated into the host genome. Examples of retroviral vectors Moloney murine leukemia virus (MLV)-derived vectors, retroviral vectors based on a Murine Stem Cell Virus, which provides long-term stable expression in target cells such as hematopoietic precursor cells and their differentiated progeny (see, e.g., Hawley et al., PNAS USA 93:10297-10302, 1996; Keller et al., Blood 92:877-887, 1998), hybrid vectors (see, e.g., Choi, et al., Stem Cells 19:236-246, 2001), and complex retrovirus-derived vectors, such as lentiviral vectors.

Examples of lentiviruses include HIV (human immunodeficiency virus; including HIV type 1, and HIV type 2), visna-maedi, the caprine arthritis-encephalitis virus, equine infectious anemia virus, feline immunodeficiency virus (FIV), bovine immune deficiency virus (BIV), and simian immunodeficiency virus (SIV). Lentiviral vectors can be derived from any one or more of these lentiviruses (see, e.g., Evans et al., Hum Gene Ther. 10:1479-1489, 1999; Case et al., PNAS USA 96:2988-2993, 1999; Uchida et al., PNAS USA 95:11939-11944, 1998; Miyoshi et al., Science 283:682-686, 1999; Sutton et al., J Virol 72:5781-5788, 1998; and Frecha et al., Blood. 112:4843-52, 2008).

Adenoviral vectors, methods for construction thereof and methods for propagating thereof, are well known in the art and are described in, for example, U.S. Pat. Nos. 9,125,870, 5,559,099, 5,837,511, 5,846,782, 5,851,806, 5,994,106, 5,994,128, 5,965,541, 5,981,225, 6,040,174, 6,020,191, and 6,113,913, and Thomas Shenk, “Adenoviridae and their Replication,” M. S. Horwitz, “Adenoviruses,” Chapters 67 and 68, respectively, in Virology, B. N. Fields et al., eds., 3d ed., Raven Press, Ltd., New York (1996).

Adeno-associated viral vectors, methods for construction thereof and methods for propagating thereof, are well known in the art and are described in, for example in U.S. Pat. Nos. 6,448,074, 8,318,687, and 8,394,386.

Vaccinia viruses have been used for decades as vectors for foreign antigens (Smith et al., Biotechnology and Genetic Engineering Reviews 2. 383-407 [1984]). Methods of inserting foreign DNA into vaccinia virus is well-known to those in the field of vaccine development and protein engineering.

Modified Vaccinia Ankara (MVA) virus is related to vaccinia virus. MVA was engineered for use as a viral vector for recombinant gene expression or as a recombinant vaccine (Sutter, G. et al. [1994], Vaccine 12:1032-40). Modified MVA for use as vaccines or other viral vector are described in U.S. Pat. Nos. 6,913,752, 6,960,345, 9,133,478 and 9,463,238.

Construction of viral vectors involves the use of standard molecular biological techniques, such as those described in, for example, Sambrook et al., Molecular Cloning, a Laboratory Manual, 2d ed., Cold Spring Harbor Press, Cold Spring Harbor, N.Y. (1989), Watson et al., Recombinant DNA, 2d ed., Scientific American Books (1992), and Ausubel et al., Current Protocols in Molecular Biology, Wiley Interscience Publishers, NY (1995), and other references mentioned herein.

Virus Like Particles typically comprise a viral polypeptide(s) derived from a structural protein(s) of a virus. As described in U.S. Pat. No. 9,051,359, methods for producing and characterizing recombinantly produced VLPs have been described based on several viruses, including influenza virus (Bright et al. (2007) Vaccine. 25:3871), human papilloma virus type 1 (Hagnesee et al. (1991) J. Virol. 67:315), human papilloma virus type 16 (Kirnbauer et al. Proc. Natl. Acad. Sci. (1992) 89:12180), HIV-1 (Haffer et al., (1990) J. Virol. 64:2653), and hepatitis A (Winokur (1991) 65:5029), and can be adapted to the viral strain of interest.

### Methods of Making Vector

The present disclosure also provides methods for making a recombinant vector described herein comprising growing a cell comprising said vector under conditions whereby the STING protein and/or cGAS protein is produced; and optionally isolating said vector.

In various embodiments, the vector is a VSV vector, optionally a replication defective VSV and the host cells comprising the VSV protein function essential for VSV replication such that said VSV vector is capable of replication in said host cell. In some embodiments, the VSV vector comprises nucleic acid encoding a STING protein and/or a cGAS protein.

Methods of making VSV vectors are described in U.S. Patent Publication 20140088177, incorporated herein by reference. Briefly, VSV mRNA can be synthesized in vitro, and cDNA prepared by standard methods, followed by insertion into cloning vectors (see, e.g., Rose and Gallione, 1981,1. Virol. 39(2):519-528). VSV or portions of VSV can be prepared using oligonucleotide restriction enzymes). Polynucleotides used for making VSV vectors herein may be obtained using standard methods in the art, such as chemical synthesis, recombinant methods and/or obtained from biological sources. Individual cDNA clones of VSV RNA can be joined by use of small DNA fragments covering the gene junctions, generated by use of reverse transcription and polymerase chain reaction (RT-PCR) from VSV genomic RNA.

VSV may be genetically modified in order to alter its properties for use in vivo. Methods for the genetic modification of VSV are well established within the art. For example, a reverse genetic system has been established for VSV (Roberts et al., Virology, 1998, 247:1-6) allowing for modifications of the genetic properties of the VSV. Standard techniques well-known to one of skill in the art may be used to genetically modify VSV and introduce desired genes within the VSV genome to produce recombinant VSVs (see for example, Sambrooke et al., 1989, A Laboratory Manual, New York: Cold Spring Harbor Laboratory Press). For insertion of nucleotide sequences into VSV vectors, for example nucleotide sequences encoding a STING protein and/or a cGAS protein, or for VSV gene sequences inserted into vectors, such as for the production helper cell lines, specific initiation signals are required for efficient translation of inserted protein coding sequences. These signals include the ATG initiation codon and adjacent sequences. In cases where an entire VSV gene, such as G-protein including its own initiation codon and adjacent sequences are inserted into the appropriate vectors, no additional translational control signals may be needed. However, in cases where only a portion of the gene sequence is inserted, exogenous translational control signals, including the ATG initiation codon, must be provided. The initiation codon must furthermore be in phase with the reading frame of the protein coding sequences to ensure translation of the entire insert. These exogenous translational control signals and initiation codons can be of a variety of origins, both natural and synthetic.

Following infection of a host cell, recombinant VSV shuts down host cell protein synthesis and expresses not only its own five gene products, but also heterologous proteins encoded within its genome. Successful expression of heterologous nucleic acid from VSV recombinants requires only the addition of the heterologous nucleic acid sequence into the full-length cDNA along with the minimal conserved sequence found at each VSV gene junction. This sequence (SEQ ID NO:18) consists of the polyadenylation/transcription stop signal (3′ AUACU7, SEQ ID NO:18, residues 1-11) followed by an intergenic dinucleotide (GA or CA) and a transcription start sequence (3′-UUGUCNNUAG, SEQ ID NO:18, residues 14-23) complementary to the 5′ ends of all VSV mRNAs. Ball et al. 1999, J. Virol. 73:4705-4712; Lawson et al. 1995, P.N.A.S. USA 92:4477-4481; Whelan et al. 1995, P.N.A.S. USA 92:8388-8392. Additionally, restriction sites, preferably unique, (e.g., in a polylinker) are introduced into the VSV cDNA, for example in intergenic regions, to facilitate insertion of heterologous nucleic acid, such as nucleic acid encoding an interleukin or interferon.

In various embodiments, the vector is a HSV vector, optionally a replication defective HSV and the host cells comprising the HSV protein function essential for HSV replication such that said HSV vector is capable of replication in said host cell. In some embodiments, the HSV vector comprises nucleic acid encoding a STING protein and/or a cGAS protein.

In other examples, the VSV (or HSV) cDNA is constructed so as to have a promoter operatively linked thereto. The promoter should be capable of initiating transcription of the cDNA in an animal or insect cell in which it is desired to produce the recombinant VSV vector (or HSV vector). Promoters which may be used include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); heat shock promoters (e.g., hsp70 for use in Drosophila S2 cells); the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol Cell Biol. 5:1639-1648; Hammer et al. 1987, Science 235:53-58; alpha 1-antitrypsin gene control region which is active in the liver (Kelsey et al., 1987, Genes and Devel. 161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); and myosin light chain-2 gene control region which is active in skeletal muscle (Sani, 1985, Nature 314:283-286). Optionally, the promoter is an MA polymerase promoter, preferably a bacteriophage or viral or insect RNA polymerase promoter, including but not limited to the promoters for T7 RNA polymerase, SP6 RNA polymerase, and T3 RNA polymerase. If an RNA polymerase promoter is used in which the RNA is not endogenously produced by the host cell in which it is desired to produce the recombinant VSV (or HSV), a recombinant source of the RNA polymerase must also be provided in the host cell. Such RNA polymerase are known in the art.

The VSV (or HSV) cDNA can be operably linked to a promoter before or after insertion of nucleic acid encoding a heterologous protein, such as a STING protein and/or a cGAS protein. In some examples, a transcriptional terminator is situated downstream of the VSV (or HSV) cDNA. In other examples, a DNA sequence that can be transcribed to produce a ribozyme sequence is situated at the immediate 3′ end of the VSV (or HSV) cDNA, prior to the transcriptional termination signal, so that upon transcription a self-cleaving ribozyme sequence is produced at the 3′ end of the antigenomic RNA, which ribozyme sequence will autolytically cleave (after a U) this fusion transcript to release the exact 3′ end of the VSV antigenomic (+)RNA. Any ribozyme sequence known in the art may be used, as long as the correct sequence is recognized and cleaved. (It is noted that hammerhead ribozyme is probably not suitable for use.)

The present disclosure provides for expression systems comprising a VSV (or HSV) vector comprising one or more heterologous nucleotide sequence(s), such as, a nucleic acid encoding a STING protein and/or a cGAS protein, inserted within a region of the VSV (or HSV) essential for replication, such as the G glycoprotein region, or other region essential for replication, such that the VSV (or HAV) lacks the essential function and is replication-defective. The VSV (or HSV) vector may have a mutation, such as a point mutation or deletion of part or all, of any region of the VSV (or HSV) genome, including the G, M, N, L or P region. If the mutation is in a region essential for replication, the VSV (or HSV) will be grown in a helper cell line that provides the essential region function. The VSV (or HSV) may also comprise a mutation, such as for example, a point mutation or deletion of part or all of a nucleotide sequence essential for replication, and optionally, with the heterologous nucleotide sequence inserted in the site of the deleted nucleotide sequence. The heterologous nucleotide sequence may be operably linked to a transcriptional regulatory sequence. Following infection of a target malignant or tumor cell, progeny viruses will lack essential protein function and cannot disseminate to infect surrounding tissue. In additional embodiments, the VSV (or HSV) vector is mutated in nucleic acid, such as by point mutation, substitution or addition of nucleic acid, or deletion of part or all, of nucleic acid encoding other VSV (or HSV) protein function such as, M protein and/or N protein function. VSV (or HSV) may be targeted to a desired site in vitro to increase viral efficiency. For example, modification of VSV G protein (or other VSV proteins) to produce fusion proteins that target specific sites may be used to enhance VSV efficiency in vivo. Such fusion proteins may comprise, for example, but not limited to single chain Fv fragments that have specificity for tumor antigens. (Lorimer et al., P.N.A.S. U.S.A., 1996. 93:14815-20).

A VSV (or HSV) vector lacking a gene(s) essential for viral replication can be grown in an appropriate complementary cell line. Accordingly, the present invention provides recombinant helper cell lines or helper cells that provide a VSV (or HSV) protein function essential for replication of a replication-deficient VSV (or HSV) construct. In some examples, the protein function is G-protein function. For example, a VSV (or HSV) vector comprising nucleic acid encoding a cytokine and lacking G-protein function can be grown in a cell line, i.e., a helper cell line, for example, a mammalian cells line such as CHO cell line, permissive for VSV (or HSV) replication, wherein said cell line expresses an appropriate G-protein function, such that said VSV (or HSV) is capable of replicating in the cell line. These complementing or helper cell lines are capable of allowing a replication-defective VSV (or HSV) to replicate and express one or more foreign genes or fragments thereof encoded by the heterologous nucleotide sequence. In some embodiments, the VSV (or HSV) vector lacks a protein host cell line comprises nucleic acid encoding the protein function essential for replication, such as for example, VSV G-protein function. Complementing cell lines can provide VSV (or HSV) viral function through, for example, co-infection with a helper virus, or by integration or otherwise maintaining in stable form part or all of a viral genome encoding a particular viral function. In other examples, additional VSV (or HSV) non-essential proteins can be deleted or heterologous nucleotide sequences inserted into nucleotide regions encoding non-essential VSV (or HSV), such as for example, the M and N proteins. The heterologous nucleotide sequence can be inserted into a region non-essential for replication wherein the VSV is replication competent. Heterologous nucleotide sequences can be inserted in non-essential regions of the VSV genome, without necessitating the use of a helper cell line for growth of the VSV vector.

The recombinant VSV (or HSV) described herein are produced for example, by providing in an appropriate host cell VSV (or HSV) cDNA wherein said cDNA comprises nucleotide sequence encoding a heterologous protein, such as for example, a STING protein and/or a cGAS protein. The nucleic acid encoding a heterologous protein can be inserted in a region non-essential for replication, or a region essential for replication, in which case the VSV (or HSV) is grown in the presence of an appropriate helper cell line. The production of recombinant VSV (or HSV) vector is carried out in vitro, in cell culture, or in cells permissive for growth of the VSV (or HSV). Standard recombinant techniques can be used to construct expression vectors containing DNA encoding VSV (or HSV) proteins. Expression of such proteins may be controlled by any promoter/enhancer element known in the art. Promoters which may be used to control expression of VSV (or HSV) proteins can be constitutive or inducible.

### Host Cells

The present invention also provides host cells comprising (i.e., transformed, transfected or infected with) the vectors or particles described herein. Both prokaryotic and eukaryotic host cells, including insect cells, can be used as long as sequences requisite for maintenance in that host, such as appropriate replication origin(s), are present. For convenience, selectable markers are also provided. Host systems are known in the art and need not be described in detail herein. Prokaryotic host cells include bacterial cells, for example, E. coli., B. subtilis, and mycobacteria. Among eukaryotic host cells are yeast, insect, avian, plant, C. elegans (or nematode) and mammalian host cells. Examples of fungi (including yeast) host cells are S. cerevisiae, Kluyveromyces lactis (K. lactis), species of Candida including C. albicans and C. glabrata, Aspergillus nidulans, SchizoSaccharomyces pombe (S. pombe), Pichia pastoris, and Yarrowia lipolytica. Examples of mammalian cells are COS cells, mouse L cells, LNCaP cells, Chinese hamster ovary (CHO) cells, human embryonic kidney (HEK) cells and African green monkey cells. Xenopus laevis oocytes, or other cells of amphibian origin, may also be used.

The present disclosure also includes compositions, including pharmaceutical compositions, containing the vector(s), immunogenic compositions, vaccines or viral particles described herein. Compositions can comprise a vector(s) described herein and a suitable solvent, such as a physiologically acceptable buffer. These are well known in the art. In other embodiments, these compositions further comprise a pharmaceutically acceptable excipient. These compositions, which can comprise an effective amount of a vector in a pharmaceutically acceptable excipient, are suitable for systemic or local administration to individuals in unit dosage forms, sterile parenteral solutions or suspensions, sterile non-parenteral solutions or oral solutions or suspensions, oil in water or water in oil emulsions and the like. Formulations for parenteral and nonparenteral drug delivery are known in the art and are set forth in Remington's Pharmaceutical Sciences, 19th Edition, Mack Publishing (1995). Compositions also include lyophilized and/or reconstituted forms of the vectors (including those packaged as a virus) of the invention.

The present disclosure also contemplates kits containing vector(s), immunogenic compositions, vaccines or viral particles described herein. These kits can be used for example for producing proteins for screening, assays and biological uses, such as a vaccine therapeutic. Procedures using these kits can be performed by clinical laboratories, experimental laboratories, medical practitioners, or private individuals.

The kits comprise a vector described herein in suitable packaging. The kit may optionally provide additional components that are useful in the procedure, including, but not limited to, buffers, developing reagents, labels, reacting surfaces, means for detection, control samples, instructions, and interpretive information. The kit may include instructions for administration of a VSV (or HSV) vector or vaccine composition.

### Methods of Use

In various embodiments, the disclosure provides a method of activating the immune system using (1) a STING protein or a constitutively active mutant of the STING protein and (2) a cGAS protein or a constitutively active cGAS protein (DCNV). Contemplated is administration of a vector comprising a nucleic acid encoding (or vaccine comprising) (1) a STING protein or a constitutively active mutant of the STING protein and (2) a cGAS protein or a constitutively active cGAS protein (DCNV) to a subject in need of immune system stimulation, e.g., induction of an immune response. In various embodiments, the immune response is an ongoing immune response in cancer. In various embodiments, the vector may be administered prophylactically in a disease or disorder in which an immune response is in a remission phase, e.g., in cancer.

In one embodiment, the disclosure provides a method of decreasing the size of a tumor in a subject having a tumor or cancer comprising administering a composition comprising (1) a STING protein or a constitutively active mutant of the STING protein and (2) a cGAS protein or a constitutively active cGAS protein (DCNV). Also provided is a method for treating cancer or preventing the recurrence of cancer comprising administering to a subject in need thereof a pharmaceutical composition comprising (1) a STING protein or a constitutively active mutant of the STING protein and (2) a cGAS protein or a constitutively active cGAS protein (DCNV), or vector comprising a nucleic acid encoding (or vaccine comprising) the (1) a STING protein or a constitutively active mutant of the STING protein and (2) a cGAS protein or a constitutively active cGAS protein (DCNV).

Exemplary conditions or disorders that can be treated with the vectors described herein include cancers, such as esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, hodgkin's lymphoma, follicular lymphoma, non-hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;ql2); PML-RARA; alkylating agent-related acute myeloid leukemia, chronic lymphocytic leukemia, richter's syndrome; waldenstrom macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent merkel cell carcinoma; stage III merkel cell carcinoma; stage IV merkel cell carcinoma, myelodysplastic syndrome and recurrent mycosis fungoides and Sezary syndrome.

In some embodiments, vectors described herein (or compositions comprising such vectors) are administered to a subject suffering from ovarian cancer, colon cancer, melanoma, breast cancer or lung cancer.

It is contemplated that the methods herein reduce tumor size or tumor burden in the subject, and/or reduce metastasis in the subject. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

It is contemplated that the methods herein reduce tumor burden, and also reduce or prevent the recurrence of tumors once the cancer has gone into remission.

In various embodiments, vectors described herein (or compositions comprising such vectors) herein modulate immune cells in a tumor. In some embodiments, the vectors described herein (or compositions comprising such vectors) increase the number of macrophages or other phagocytes or antigen presenting cells in a tumor and/or increases phagocytic activity of cells in the area of the tumor.

### Methods of Administration

Many methods may be used to administer or introduce (1) a STING protein or a constitutively active mutant of the STING protein and (2) a cGAS protein or a constitutively active cGAS protein (DCNV), or vectors, vaccines or viral particles comprising a nucleic acid encoding (1) a STING protein or a constitutively active mutant of the STING protein and (2) a cGAS protein or a constitutively active cGAS protein (DCNV) into individuals (i.e., including subjects or patients), including but not limited to, intratumorally, intravenously, intra-arterially, intraperitoneally, intranasally, intramuscularly, intradermally, subcutaneously, orally or by continuous infusion.

The individual to which a vector or viral particle described herein is administered is a primate, or in other examples, a mammal, or in other examples, a human, but can also be a non-human mammal including but not limited to cows, horses, sheep, pigs, fowl, cats, dogs, hamsters, mice and rats. In the use of a vector, vaccines or viral particles, the individual can be any animal in which a vector or virus is capable introducing the (1) STING protein or a constitutively active mutant of the STING protein and (2) the cGAS protein or a constitutively active cGAS protein (DCNV) and results in activation of the immune response.

The present invention encompasses compositions comprising (1) a STING protein or a constitutively active mutant of the STING protein and (2) a cGAS protein or a constitutively active cGAS protein (DCNV), or vectors, vaccines or viral particles can further comprise a pharmaceutically acceptable carrier. The amount of vector(s) to be administered will depend on several factors, such as route of administration, the condition of the individual, the degree of aggressiveness of the malignancy, and the particular vector employed. Also, the vector may be used in conjunction with other treatment modalities.

The dose of vector, vaccine or viral particle to be employed in the formulation will also depend on the route of administration, and the nature of the patient, and should be decided according to the judgment of the practitioner and each patient's circumstances according to standard clinical techniques. The exact amount of vector or virus utilized in a given preparation is not critical provided that the minimum amount of virus necessary to produce immunologic activity is given.

Effective doses of the vector, vaccine or viral particle of the disclosure may also be extrapolated from dose-response curves derived from animal model test systems.

If administered as a viral vector(s), vaccines or viral particles from about 10 up to about 10⁷ p.f.u., in other examples, from about 10³ up to about 10⁶ p.f.u., and in other examples, from about 10⁴ up to about 10⁵ p.f.u. is administered. If administered as a polynucleotide construct (i.e., not packaged as a virus), about 0.01 μg to about 100 μg of a viral construct of the present invention can be administered, in other examples, 0.1 μg to about 500 μg, and in other examples, about 0.5 μg to about 200 μg can be administered. More than one vector, vaccine or viral particlewcan be administered, either simultaneously or sequentially. Administrations are typically given periodically, while monitoring any response. Administration can be given, for example, intramuscularly, intravenously, intratumorally or intraperitoneally.

It is contemplated that an effective amount of the (1) STING protein or a constitutively active mutant of the STING protein and (2) cGAS protein or a constitutively active cGAS protein (DCNV), or vectors, vaccines or viral particles, vector(s), vaccines or viral particles is administered. An “effective amount” is an amount sufficient to achieve a desired biological effect such as to induce enough humoral or cellular immunity. This may be dependent upon the type of vaccine, the age, sex, health, and weight of the recipient. Examples of desired biological effects include, but are not limited to, increase in immune response, increase in STING stimulation, decrease in tumor size or tumor burden, production of no symptoms or reduction in symptoms related to disease or condition being treated.

A vaccine or composition of the present invention is physiologically significant if its presence results in a detectable change in the physiology of a recipient patient that enhances at least one primary or secondary humoral or cellular immune response against a tumor or other targeted cell or microbe. For example, in certain embodiments, the (1) a STING protein or a constitutively active mutant of the STING protein and (2) a cGAS protein or a constitutively active cGAS protein (DCNV), or vectors, vaccines or viral particles increases infiltration of immune cells into the tumor or site of infection. In certain embodiments, the immune cells are macrophages, dendritic cells or other phagocytes.

The composition, if desired, can also contain minor amounts of wetting or emulsifying agents or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water free concentrate in a hermetically sealed container such as an ampoule or sachet indicating the quantity of active agent. Where the composition is administered by injection, an ampoule of sterile diluent can be provided so that the ingredients may be mixed prior to administration.

Pharmaceutical compositions of the present disclosure containing the vector, vaccine or viral particle described herein as an active ingredient may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present disclosure.

Formulation of the pharmaceutical composition will vary according to the route of administration selected (e.g., solution, emulsion). An appropriate composition comprising the vector, vaccine or viral particle, to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Therapeutic formulations of the vector, vaccine or viral particle are prepared for storage by mixing the vector, vaccine or viral particle having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

In one embodiment, administration is performed at the site of a cancer or affected tissue needing treatment by direct injection into the site or via a sustained delivery or sustained release mechanism, which can deliver the formulation internally. For example, biodegradable microspheres or capsules or other biodegradable polymer configurations capable of sustained delivery of a composition can be included in the formulations of the disclosure implanted near or at site of the cancer.

Therapeutic compositions may also be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, every other day, twice weekly, three times weekly, weekly, every 2 weeks, every 3 weeks, monthly, or at a longer interval.

### Combination Therapy

It is contemplated that a vector or vaccine of the present disclosure or composition thereof is administered with a second agent useful for treating a condition or disorder, e.g., cancer.

Concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks.

The second agent may be other therapeutic agents, such as cytokines, growth factors, other inhibitors and antibodies to target antigens useful for treating cancer or immunological disorders, for example ipilimumab (YERVOY®, Bristol-Myers Squibb Company), an antibody to CTLA-4; bevacizumab (AVASTIN®, Genentech), an antibody to VEGF-A; erlotinib (TARCEVA®, Genentech and OSI Pharmaceuticals), a tyrosine kinase inhibitor which acts on EGFR, dasatinib (SPRYCEL®, Bristol-Myers Squibb Company), an oral Bcr-Abl tyrosone kinase inhibitor; IL-21; pegylated IFN-oc2b; axitinib (INLYTA®, Pfizer, Inc.), a tyrosine kinase inhibitor; and trametinib (MEKINIST®, GlaxoSmithKline), a MEK inhibitor (Philips and Atkins, Int Immunol., 27(I):39-46 (2015) which is incorporated herein by reference).

It is contemplated that the vectors disclosed herein (or compositions comprising such vectors) and second agent may be given simultaneously, in the same formulation. It is further contemplated that the vectors disclosed herein (or compositions comprising such vectors), and second agent are administered in a separate formulation and administered concurrently, with concurrently referring to agents given within 30 minutes of each other.

In another aspect, the vectors disclosed herein (or compositions comprising such vectors) are administered prior to administration of the second agent. Prior administration refers to administration of the vectors disclosed herein (or compositions comprising such vectors) within the range of one week prior to treatment with the second agent, up to 30 minutes before administration of the second agent. It is further contemplated that the vectors disclosed herein (or compositions comprising such vectors) are administered subsequent to administration a second agent. Subsequent administration is meant to describe administration from 30 minutes after administration of the vectors disclosed herein (or compositions comprising such vectors) up to one week after administration of the vectors disclosed herein (or compositions comprising such vectors).

It is further contemplated that other adjunct therapies may be administered, where appropriate. For example, the patient may also be administered surgical therapy, chemotherapy, a cytotoxic agent, photodynamic therapy or radiation therapy where appropriate.

It is further contemplated that when the vectors disclosed herein (or compositions comprising such vectors) are administered in combination with a second agent, such as for example, wherein the second agent is a cytokine or growth factor, or a chemotherapeutic agent, the administration also includes use of a radiotherapeutic agent or radiation therapy. The radiation therapy administered in combination with composition described herein is administered as determined by the treating physician, and at doses typically given to patients being treated for cancer.

A cytotoxic agent refers to a substance that inhibits or prevents the function of cells and/or causes destruction of cells. The term is intended to include radioactive isotopes (e.g., I131, I125, Y90 and Rel86), chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A non-cytotoxic agent may include an agent that can be activated to be cytotoxic. A non-cytotoxic agent may include a bead, liposome, matrix or particle (see, e.g., U.S. Patent Publications 2003/0028071 and 2003/0032995 which are incorporated by reference herein).

Chemotherapeutic agents contemplated for use with the vectors, vaccines, or viral particles of the present disclosure include, but are not limited to those listed in Table I:

TABLE I

| | |
|---|---|
| Alkylating agents | Natural products |
| Nitrogen mustards | Antimitotic drugs |
| mechlorethamine | Taxanes |
| cyclophosphamide | paclitaxel |
| ifosfamide | Vinca alkaloids |
| melphalan | vinblastine (VLB) |
| chlorambucil | vincristine |
| Nitrosoureas | vinorelbine |
| carmustine (BCNU) | Taxotere ® (docetaxel) |
| lomustine (CCNU) | estramustine |
| semustine (methyl-CCNU) | estramustine phosphate |
| Ethylenimine/Methyl-melamine | Epipodophylotoxins |
| thriethylenemelamine (TEM) | etoposide |
| triethylene thiophosphoramide (thiotepa) | teniposide |
| hexamethylmelamine (HMM, altretamine) | Antibiotics |
| Alkyl sulfonates | actimomycin D |
| busulfan | daunomycin (rubido-mycin) |
| Triazines | doxorubicin (adria-mycin) |
| dacarbazine (DTIC) | mitoxantroneidarubicin |
| Antimetabolites | bleomycin |
| Folic Acid analogs | splicamycin (mithramycin) |
| methotrexate | mitomycinC |
| Trimetrexate | dactinomycin |
| Pemetrexed (Multi-targeted antifolate) | aphidicolin |
| Pyrimidine analogs | Enzymes |
| 5-fluorouracil | L-asparaginase |
| fluorodeoxyuridine | L-arginase |
| gemcitabine | Radiosensitizers |
| cytosine arabinoside (AraC, cytarabine) | metronidazole |
| 5-azacytidine | misonidazole |
| 2,2′-difluorodeoxy-cytidine | desmethylmisonidazole |
| Purine analogs | pimonidazole |
| 6-mercaptopurine | etanidazole |
| 6-thioguanine | nimorazole |
| azathioprine | RSU 1069 |
| 2′-deoxycoformycin (pentostatin) | EO9 |
| erythrohydroxynonyl-adenine (EHNA) | RB 6145 |
| fludarabine phosphate | SR4233 |
| 2-chlorodeoxyadenosine (cladribine, 2-CdA) | nicotinamide |
| Type I Topoisomerase Inhibitors | 5-bromodeozyuridine |
| camptothecin | 5-iododeoxyuridine |
| topotecan | Bromodeoxycytidine |
| irinotecan | Miscellaneous agents |
| Biological response modifiers | Platinium coordination complexes |
| G-CSF | cisplatin |
| GM-CSF | Carboplatin |
| Differentiation Agents | oxaliplatin |
| retinoic acid derivatives | Anthracenedione |
| Hormones and antagonists | mitoxantrone |
| Adrenocorticosteroids/antagonists | Substituted urea |
| prednisone and equivalents | hydroxyurea |
| dexamethasone | Methylhydrazine derivatives |
| ainoglutethimide | N-methylhydrazine (MIH) |
| Progestins | procarbazine |
| hydroxyprogesterone caproate | Adrenocortical suppressant |
| medroxyprogesterone acetate | mitotane (o,p′-DDD) |
| megestrol acetate | ainoglutethimide |
| Estrogens | Cytokines |
| diethylstilbestrol | interferon (a, β, γ) |
| ethynyl estradiol/equivalents | interleukin-2 |
| Antiestrogen | Photosensitizers |
| tamoxifen | hematoporphyrin derivatives |
| Androgens | Photofrin ® |
| testosterone propionate | benzoporphyrin derivatives |
| fluoxymesterone/equivalents | Npe6 |
| Antiandrogens | tin etioporphyrin (SnET2) |
| flutamide | pheoboride-a |
| gonadotropin-releasing | bacteriochlorophyll-a |
| hormone analogs | naphthalocyanines |
| leuprolide | phthalocyanines |
| Nonsteroidal antiandrogens | zinc phthalocyanines |
| flutamide | Radiation |
| | X-ray |
| | ultraviolet light |
| | gamma radiation |
| | visible light |
| | infrared radiation |
| | microwave radiation |

### Kits

As an additional aspect, the disclosure includes kits which comprise one or more compounds or compositions packaged in a manner which facilitates their use to practice methods of the disclosure. In one embodiment, such a kit includes a compound or composition described herein (e.g., a composition comprising a vector, vaccine or viral particle described herein alone or in combination with another vector, vaccine or viral particle or a third agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the vector, vaccine or viral particle compositions.

Additional aspects and details of the disclosure will be apparent from the following examples, which are intended to be illustrative rather than limiting.

### EXAMPLES

### Example 1—Generation of rVSV Expressing DncV-HA and Ecuador Mutant STING (R284S)

Human STING in pCDNA-hSTING plasmid was mutated into R284S STING using QuickChange II XL site directed mutagenesis kit (Stratagene) with the following primers;

hSTING_R284S-F:
(SEQ ID NO: 3)
TTTAGCCGGGAGGATTCTCTTGAGCAGGCCAAA;
hSTING_R284S-R:
(SEQ ID NO: 4)
TTTGGCCTGCTCAAGAGAATCCTCCCGGCTAAA.

The R284S STING gene was then PCR amplified from the pCDNA plasmid using primers Forward XhoI STING 5′ CTAGCTCGAGatgccccactccagcctg (SEQ ID NO:5) and Reverse NheI STING 5′CTAGGCTAGCTCAAGAGAAATCCGTGCG (SEQ ID NO:6) to insert restriction sites XhoI and NheI flanking R284S STING. R284S STING was then cloned into pVSV-XN2 after restriction enzyme digestion with XhoI and NheI (NEB) using Electroligase (NEB).

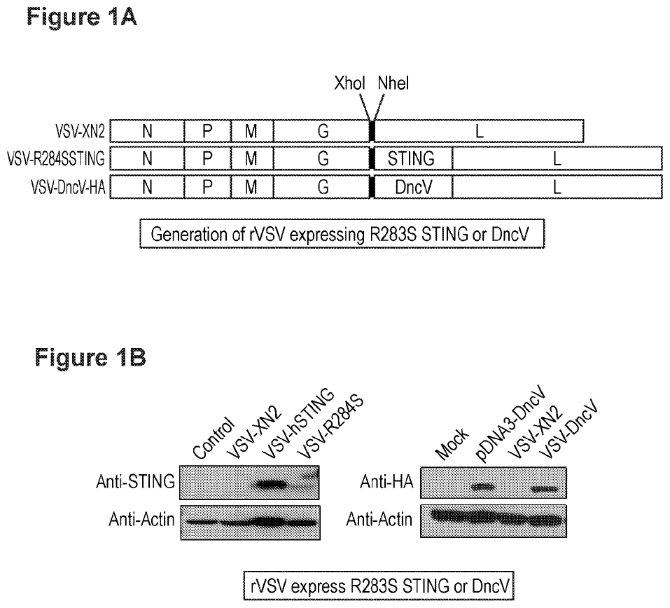

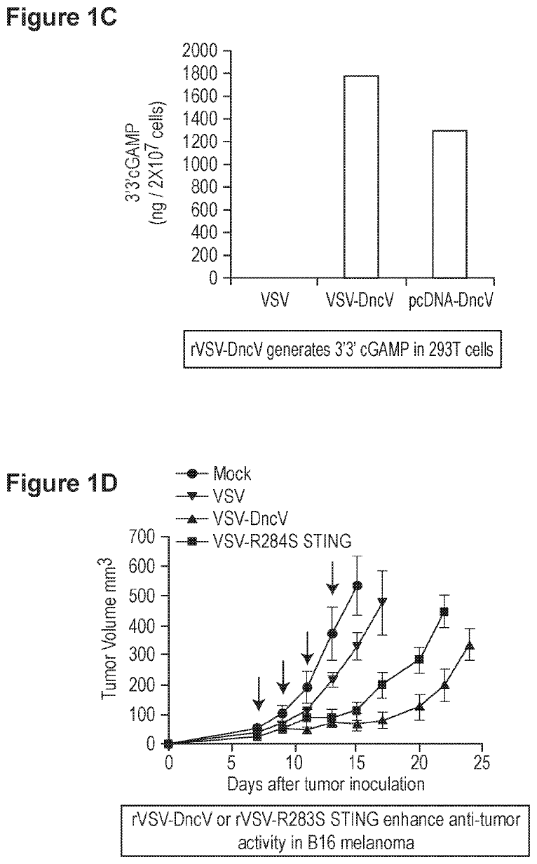

Bacterial DNCV was ordered from Genescript with codon optimization for human host expressions and a HA C terminal tag. The restriction sites XhoI and NheI are flanking DNCV-HA at the 5′ and 3′ ends, respectively. DNCV-HA was cloned into pVSV-XN2 similarly as R284S STING. FIG. 1A shows the generation of rVSV expressing R283S STING or DncV. FIG. 1B shows that the rVSV expresses R283S STING or DncV

### Example 2—Recovery of Infectious rVSV-DncV-HA and rVSV-R284S STING

Infectious virus of VSV-DNCV-HA and VSV-R284S STING was recovered using am established reverse genetics approach. In brief, 293T cells were infected with VTF7-3 vaccinia expressing T7 polymerase. After the vaccinia inoculation, the vaccinia was removed and replaced with DMEM 5% low-IgG FBS (Life Technologies). The infected 293Ts were then transfected using Lipofectamine 2000 with the following plasmids expressing VSV protein N, P, and L: 0.5 μg of pBlueScript SK (pBS)-N, 0.83 μg of pBS-P, 0.17 μg of pBS-L, and 5 μg of the respective full length rVSV plasmid. The following day the media was collected and filtered through a 0.2 pm syringe filter and plated onto a 2nd plate of 293Ts to remove residual vaccinia. If CPE was observed, the media was collected and plaques were isolated using a standard plaque assay. The plaques were then amplified once expression of the transgene was verified and VSV was purified using ultracentrifugation at 27K/4° C./90 min using a soft cushion of 10% Optiprep. The pelleted VSV was resuspended in PBS and stored at −80° C. until needed.

### Example 3—Evaluation of 3′3 cGAMP Generation by Mass Spec Analysis

The cells were infected with rVSV-DncV at MOI=0.001 for 24 hours. The infected cells were pelleted and snap-frozen in liquid nitrogen and stored at −80° C. before further processing. To extract cGAMP, frozen cells were thawed on ice and lysed in cold 80% (vol/vol) methanol with 2% (vol/vol) acetic acid (HAc). Cyclic-di-GMP was supplemented as internal standard. Cell lysates were cleared by centrifugation at 4° C., 10,000×g for 10 min. Pellets were further extracted in 20% (vol/vol) methanol with 2% HAc twice and all extracts were pooled. cGAMP was then enriched by solid-phase extraction (SPE) using HyperSep Aminopropyl SPE Columns (Thermo Scientific) as previously described in Gao et al, 2015. Briefly, columns were activated by 100% methanol and washed twice with 2% HAc; after drawing through the extracts, columns were washed twice with 2% HAc and once with 80% methanol, and finally eluted with 2% (vol/vol) ammonium hydroxide in 80% methanol. The eluents were spin-vacuumed to dryness, reconstituted in liquid chromatography (LC)/MS-grade water and stored at −20° C. before subject to LC/MS analysis. Chromatography was performed using a Thermo Scientific Surveyor MS Pump Plus pump and Micro AS autosampler. The separation was isocratic on a Water's XBridge Amide column (3.5 μm, 2.1×100 mm) at 200 μl/min using 18:82 water:acetonitrile 6.3 mM ammonium hydroxide and 6.3 mM ammonium bicarbonate. The samples were introduced into a Thermo Scientific LTQ-FT, a hybrid mass spectrometer consisting of a linear ion trap and a Fourier transform ion cyclotron resonance mass spectrometer. The standard electrospray source was used operated in negative ion mode. cGAMP was quantitated using the m/z 522 product ion from the collision-induced dissociation of the deprotonated parent ion at m/z 673. An external calibration curve derived from eight standards was used in the quantitation and acquired before and after the samples were analyzed. The c-di-GMP component was quantitated from the m/z 344 product ion originating from the deprotonated m/z 689 parent. FIG. 1C shows that vector comprising constitutively active cGAS induced the highest level of CDN.

### Example 4—Anti-Tumor Effects of rVSV-DncV or rVSV-R283 STING

For anti-tumor effects, mice were subcutaneously injected with 5×10⁵ cells of B16-OVA on the right flank. One week later, when tumors were 50 mm³ in volume, the mice were intratumorally injected with VSV, VSV-R284 STING, or VSV-DncV (2×10⁶ pfu/mouse). The tumor volume was measured using calipers and calculated with the formula V=(length×width2)/2. As shown in FIG. 1D, shows that rVSV-DncV or rVSV-R283S STING enhance anti-tumor activity in B16 melanoma.

### Example 5—Plasmid Construction and Expression of rHSV1

STING, cGAS or STING-2A-cGAS genes were inserted into the multiple cloning site of the pTransfer plasmid by ligation. For STING-2A-cGAS gene, 2A self-cleavage peptide sequences from porcine teschovirus-1 were used between STING and cGAS genes (GGAAGCGGAGCTACTAACTTCAGCCTGCTGAAGCAGGCTGGAGACGTGGAGGAGA ACCCTGGACCT (SEQ ID NO:7)). To make pTransfer-STING, pTransfer-cGAS and pTransfer-STING-2A-cGAS clones, AccuPrime Pfx SuperMix (Invitrogen) was used for PCR amplification.

The following primer pairs were used for PCR:

| | | | | SEQ |
|---|---|---|---|---|
| | | | | ID |
| | | | | NO: |
| pTransfer-STING | | Forward | 5′-GATCACTAGTATGCCCC | 8 |
| | | | ACTCCAGCCTGCAT-3′ | |
| | | Reverse | 5′-GATCCTCGAGTCAAGAG | 9 |
| | | | AAATCCGTGCGGAGAGG-3′ | |
| pTransfer-cGAS | | Forward | 5′-GATCACTAGTATGCAGC | 10 |
| | | | CTTGGCACGGAAAGGCC-3′ | |
| | | Reverse | 5′-GATCCTCGAGTCAAAAT | 11 |
| | | | TCATCAAAAACTGG-3′ | |
| pTransfer- | 1^{st} | Forward | 5′-GATCACTAGTATGCCCC | 12 |
| STING- | step | | ACTCCAGCCTGCAT-3′ | |
| 2A-cGAS | PCR | Reverse | 5′-CTCCACGTCTCCAGCCT | 13 |
| | | | GCTTCAGCAGGCTGAAGTTA | |
| | | | GTAGCTCCGCTTCCAGAGAA | |
| | | | ATCCGTGCGGA-3′ | |
| | 2^{nd} | Forward | 5′-GGAAGCGGAGCTACTAA | 14 |
| | step | | CTTCAGCCTGCTGAAGCAGG | |
| | PCR | | CTGGAGACGTGGAGGAGAAC | |
| | | | CCTGGACCTATGCAGCCTTG | |
| | | | GCACGG-3′ | |
| | | Reverse | 5-GATCCTCGAGTCAAAATT | 15 |
| | | | CATCAAAAACTGG | |
| | 3^{rd} | Forward | 5′-GATCACTAGTATGCCCC | 16 |
| | step | | ACTCCAGCCTGCAT-3′ | |
| | PCR | Reverse | 5′-GATCCTCGAGTCAAAAT | 17 |
| | | | TCATCAAAAACTGG-3′ | |

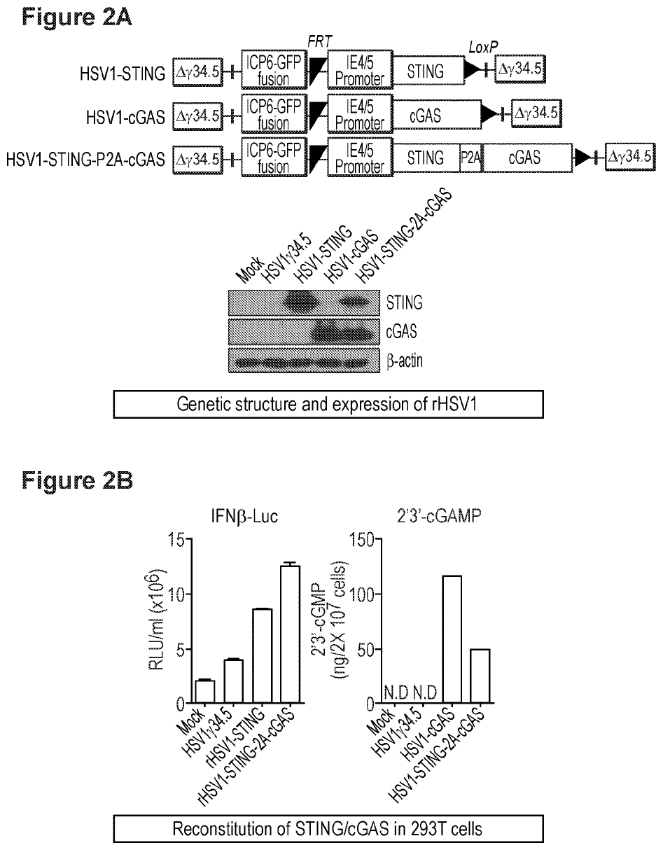

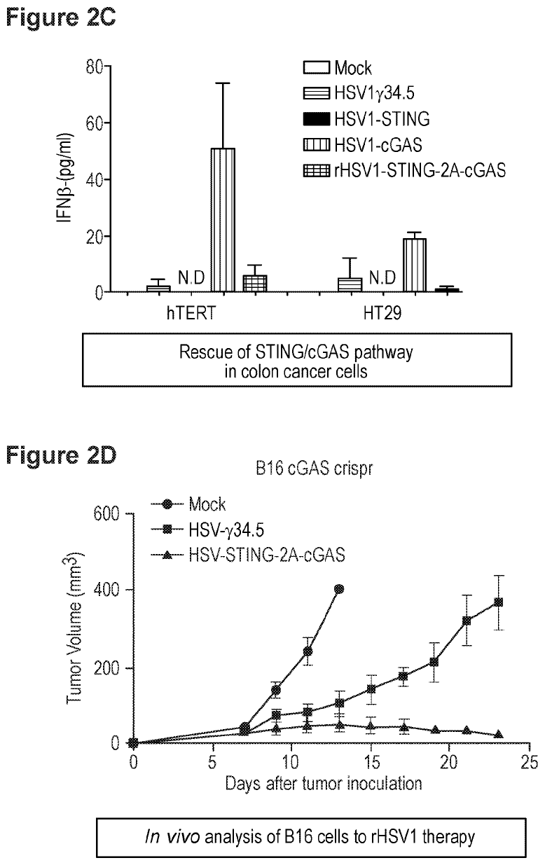

pTransfer plasmids containing STING, cGAS or STING-2A-cGAS were inserted into the FRT site of the fHSVQuik-1 BAC plasmid through Flp-mediated site-specific recombination in bacteria (FIG. 2A). The STING, cGAS or STING-2A-cGAS containing HSV recombinants were rescued by co-transfection with co-integrated HSV BAC DNA and Cre-expressing helper plasmid into Vero cells.

Virus was amplified in 293T cells. 293T cells were infected at MOI 0.1 with HSV1-γ34.5, HSV1-STING, HSV1-cGAS or HSV1-STING-2A-cGAS. Cells were collected after 48 hours infection and subjected to sonication three times. Supernatants containing virus were centrifuged at 27,000 rpm for 90 min at 4° C. through sucrose gradient cushion. Pelleted viruses were suspended in PBS and stored at −80° C. Virus titers were determined by plaque assay on Vero cells.

Immunoblot of STING, cGAS expression in HSV1-STING, HSV1-cGAS or HSV1-STING-2A-cGAS infected 293T cells was assessed. 293T cells were infected at MOI 5 with HSV1-γ34.5, HSV1-STING, HSV1-cGAS or HSV1-STING-2A-cGAS. After 6 hours infection, infected cells were collected and whole cell lysates were resolved by SDS-PAGE and transferred to polyvinylidene fluoride (PVDF) membranes. After blocking with 5% blocking buffer, membrane was incubated with rabbit-anti-STING antiserum, rabbit-anti-cGAS antibody (Cell signaling technology) or mouse anti-beta actin antibody (Sigma).

### Example 6—Reconstitution of STING/cGAS in 293T Cells

IFNβ luciferase assay: 293T cells were transfected with 50 ng IENβ-Luc plasmid and 10 ng pRL-TK (Renilla) normalization plasmid. After 24 hours transfection, 293T cells were infected at MOI of 5 with HSV1-γ34.5, HSV1-STING or HSV1-STING-2A-cGAS. IENβ promoter activities were analyzed by luminometer after 6 hours infection.

Measurement of CDN (2′3′-cGAMP): 293T cells were infected with HSV1 γ34.5, HSV1-cGAS or HSV1-STING-2A-cGAS at MOI 1. The infected cells were pelleted and snap-frozen in liquid nitrogen and stored at −80° C. before further processing. To extract cGAMP, frozen cells were thawed on ice and lysed in cold 80% (vol/vol) methanol with 2% (vol/vol) acetic acid (HAc). Cyclic-di-GMP was supplemented as internal standard. Cell lysates were cleared by centrifugation at 4° C., 10,000×g for 10 min. Pellets were further extracted in 20% (vol/vol) methanol with 2% HAc twice and all extracts were pooled. cGAMP was then enriched by solid-phase extraction (SPE) using HyperSep Aminopropyl SPE Columns (Thermo Scientific) as previously described in Gao et al, 2015. Briefly, columns were activated by 100% methanol and washed twice with 2% HAc; after drawing through the extracts, columns were washed twice with 2% HAc and once with 80% methanol, and finally eluted with 2% (vol/vol) ammonium hydroxide in 80% methanol. The eluents were spin-vacuumed to dryness, reconstituted in liquid chromatography (LC)/MS-grade water and stored at −20° C. before subject to LC/MS analysis. Chromatography was performed using a Thermo Scientific Surveyor MS Pump Plus pump and Micro AS autosampler. The separation was isocratic on a Water's XBridge Amide column (3.5 μm, 2.1×100 mm) at 200 μl/min using 18:82 water:acetonitrile 6.3 mM ammonium hydroxide and 6.3 mM ammonium bicarbonate. The samples were introduced into a Thermo Scientific LTQ-FT, a hybrid mass spectrometer consisting of a linear ion trap and a Fourier transform ion cyclotron resonance mass spectrometer. The standard electrospray source was used operated in negative ion mode. cGAMP was quantitated using the m/z 522 product ion from the collision-induced dissociation of the deprotonated parent ion at m/z 673. The c-di-GMP component was quantitated from the m/z 344 product ion originating from the deprotonated m/z 689 parent. FIG. 2B shows that STING/cGAS can be reconstituted by transfection of vector into cells that lack expression of these proteins.

### Example 7—Rescue of STING/cGAS Pathway in Colon Cancer Cells

IFNβ ELISA: hTERT-BJI telomerase normal fibroblasts (hTERT) and human colon cancer cell line HT29 cells were infected at MOI 1 with HSV1 γ34.5, HSV1-STING, HSV1-cGAS or HSV1-STING-2A-cGAS. After 24 hours infection, IFNβ level in supernatants was measured by enzyme-linked immunosorbent assay kit (PBL Assay Science). FIG. 2C shows the rescue of STING/cGAS pathway in colon cancer cells.

### Example 8—In Vivo Analysis of B16 Cells and rHSV1 Therapy

B16-OVA cGAS crispr cells (5E5 cells per mouse) were injected subcutaneously into the right flank of C57/BL6 mice. When tumor diameter reached ˜0.5 cm, the mice were injected intratumorally with HSV1 γ34.5, HSV1-STING-2A-cGAS or PBS at day 7 and 10 after tumor cell administration (1×10⁷ plaque forming unit per mouse).

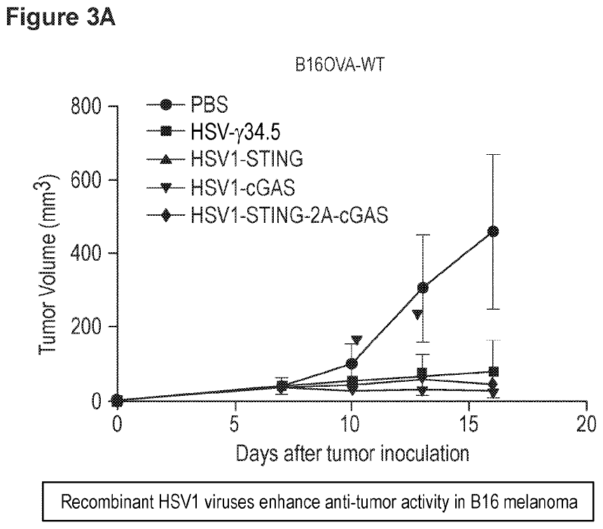

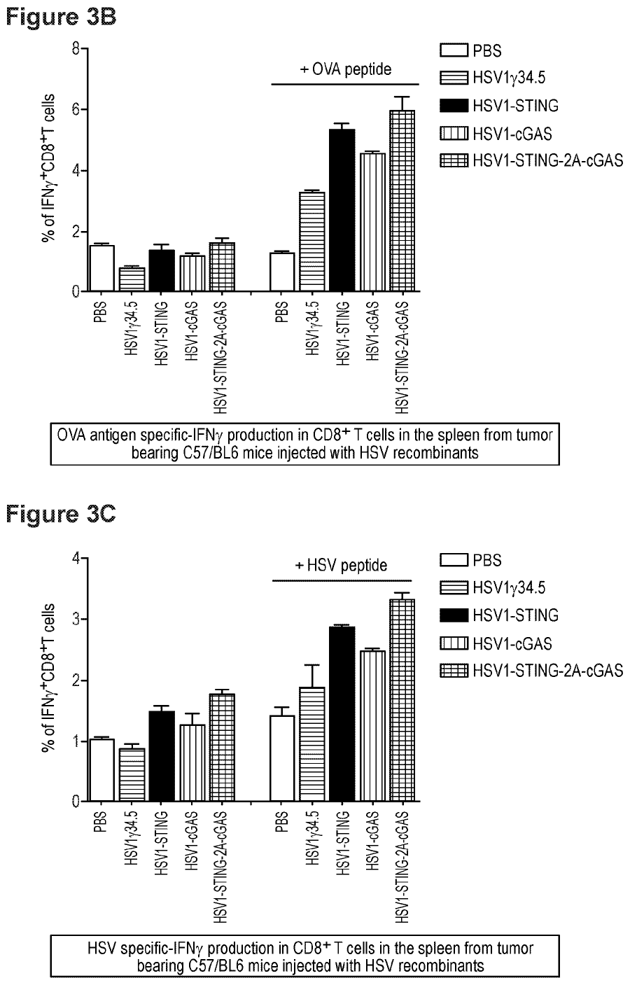

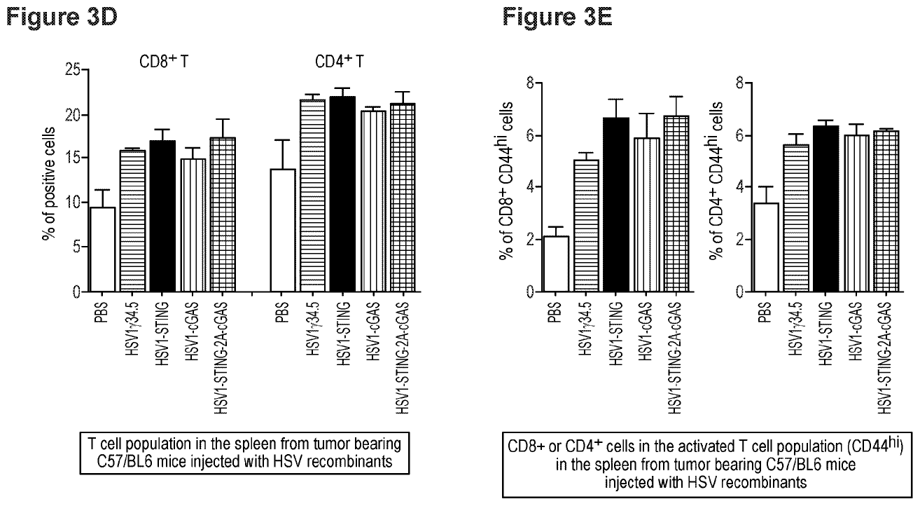

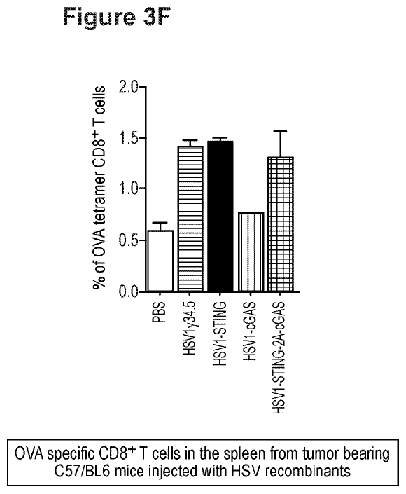

Results are provided FIG. 2D and FIGS. 3A-3F. The tumor growth was measured every other day and tumor volume was calculated with the formula V=(length×width2)/2. As shown in FIGS. 2D and 3A, recombinant HSV1 enhanced anti-tumor activity in B16 melanoma cells compared to the control vectors.

OVA antigen- and HSV-specific IFNy production in CD8+ T cells in the spleen was the highest in mice receiving the HSV1-STING-2A-cGAS treatment. See FIGS. 3B and 3C, respectively.

CD4+ and CD8+ T cell populations in spleens from tumor bearing C57/B6 mice injected with rHSV1 were analyzed, the results of which are provided in FIGS. 3D-3F.

Embodiment P1. In an embodiment, a vector comprising a human STimulator of INterferon Genes (STING) polynucleotide encoding a STING protein and a cyclic GMP-AMP synthase (Cgas) polynucleotide encoding a cGAS protein.

Embodiment P2. The vector of embodiment P1, wherein the STING protein is a constitutively active STING protein.

Embodiment P3. The vector of embodiment P2, wherein the constitutively active STING results from a mutation at amino acid 284 of SEQ ID NO:1, optionally wherein the mutation is R284S of SEQ ID NO:1.

Embodiment P4. The vector of embodiment P1, wherein the cGAS protein is a constitutively active cGAS protein.

Embodiment P5. The vector of embodiment P4, wherein the constitutively active cGAS protein is DCNV.

Embodiment P6. The vector of embodiment P1, wherein the vector comprises a polynucleotide encoding a constitutively active STING protein and a constitutively active cGAS protein.

Embodiment P7. The vector of embodiment P1, wherein the vector comprises a polynucleotide encoding a STING protein and a constitutively active cGAS protein.

Embodiment P8. The vector of embodiment P1, wherein the vector comprises a polynucleotide encoding a constitutively active STING protein and a cGAS protein.

Embodiment P9. The vector of any one of embodiment P1-P8, wherein the vector is a viral or plasmid vector.

Embodiment P10. The vector of embodiment P9, wherein the viral vector is selected from the group consisting of vesicular stomatitis virus (VSV), a herpes simplex virus (HSV), a lentivirus, an adenovirus, an adeno-associated virus, a vaccinia virus and a modified vaccinia Ankara (MVA) virus.

Embodiment P11. The vector of any one of embodiment P1-P10, wherein the vector is a VSV vector.

Embodiment P12. The vector of any one of embodiment P1-P10, wherein the vector is a HSV-1 vector.

Embodiment P13. A composition comprising the vector of any one of embodiment P1-P12.

Embodiment P14. A method of stimulating an immune response in a subject in need thereof comprising administering a composition of embodiment P13 to the subject, wherein the composition induces STING signaling.

Embodiment P15. The method of embodiment P14, wherein the subject is suffering from cancer.

Embodiment P16. A method of treating cancer in a subject comprising administering the composition of embodiment P13 to the subject, wherein the composition induces STING signaling.

Embodiment P17. The method of embodiment P15 or embodiment P16, wherein the cancer is ovarian cancer, colon cancer, melanoma, breast cancer or lung cancer.

Embodiment P18. The method of embodiment P15 or embodiment P16, wherein tumor size in the subject is decreased by about 25-50%, about 40-70% or about 50-90% or more.

Embodiment P19. The method of any one of embodiment P14-P18, wherein the composition is administered intratumorally, intravenously, intra-arterially, intraperitoneally, intranasally, intramuscularly, intradermally or subcutaneously.

Embodiment P20. The method of any one of embodiment P14-P19, wherein the composition induces infiltration of immune cells into the tumor.

Embodiment P21. The method of embodiment P20, wherein the immune cells are macrophages or other phagocytes.

What is claimed:

1. A method of treating a cancer in a mammal suffering from the cancer comprising:
(A) introducing into the mammal a recombinant VSV vector (Vesicular Stomatitis Virus vector) which is a first template for synthesis of a VSV protein function, where the recombinant VSV vector is capable of replication in a host cell, where the recombinant VSV vector encodes a mammalian STING polynucleotide (STimulator of INterferon Genes polynucleotide), where the mammalian STING polynucleotide is expressed by the recombinant VSV vector, where the mammalian STING polynucleotide is a second template for synthesis of a STING protein, where the recombinant VSV vector further comprises:
(a) a first substitution, where the first substitution causes the mammalian STING polynucleotide to be constitutively active in the mammal;
(b) a second substitution, where the second substitution is to a VSV G gene, where the second substitution generates a VSV G stem mutation, where expression of the VSV G gene in the mammal is inhibited by the VSV G stem mutation; and
(B) introducing a PEG-ated-IFN (polyethylene glycol-ated interferon) therapeutic agent in the mammal.

2. The method of claim 1, where the first substitution is a mutation at amino acid 154 of SEQ ID NO:1.

3. The method of claim 2, where the mutation is N154S.

4. The method of claim 1, where the recombinant VSV vector encodes a VSV genome, where the VSV genome has an order 3′-NPMGL-5′.

5. The method of claim 1, where the recombinant VSV vector further comprises a cGAS polynucleotide (cyclic GMP-AMP synthase polynucleotide) which is a third template for synthesis of a cGAS protein.

6. The method of claim 5, where the cGAS protein is constitutively active.

7. The method of claim 1, where the mammal is suffering from a cancerous tumor.

8. The method of claim 7, where the mammal is a human subject and the mammalian STING polynucleotide is human STING polynucleotide.

9. The method of claim 8, where the cancerous tumor is selected from the group consisting of an ovarian cancer tumor, a colon cancer tumor, a melanoma tumor, a breast cancer tumor, and a lung cancer tumor.

10. The method of claim 8, where the cancerous tumor is reduced in size by 50-90% or more.

11. The method of claim 1, further comprising administering an adjuvant.

12. The method of claim 1, where the recombinant VSV vector is administered intratumorally, intravenously, intra-arterially, intraperitoneally, intranasally, intramuscularly, intradermally or subcutaneously to the host cell.

13. The method of claim 1, where the PEG-ated-IFN is administered intratumorally, intravenously, intra-arterially, intraperitoneally, intranasally, intramuscularly, intradermally or subcutaneously to the mammal.

14. A method of treating a cancer in a mammal suffering from the cancer comprising:
(A) introducing into the mammal a recombinant VSV vector (Vesicular Stomatitis Virus vector) which is a first template for synthesis of a VSV protein, where the recombinant VSV vector is replication-defective, where the recombinant VSV vector encodes a mammalian STING polynucleotide (STimulator of INterferon Genes polynucleotide), where the mammalian STING polynucleotide is expressed by the recombinant VSV vector, where the mammalian STING polynucleotide is a second template for synthesis of a STING protein, where the recombinant VSV vector further comprises:
(a) a first substitution, where the first substitution causes the mammalian STING polynucleotide to be constitutively active in the mammal;
(b) a second substitution, where the second substitution is to a VSV G gene, where the second substitution generates a VSV G stem mutation, where expression of the VSV G gene in the mammal is inhibited by the VSV G stem mutation; and
(B) introducing a PEG-ated-IFN (polyethylene glycol-ated interferon) therapeutic agent in the mammal.

15. The method of claim 14, where the first substitution is a mutation at amino acid 154 of SEQ ID NO:1, where asparagine is replaced with serine.

16. The method of claim 14, where the recombinant VSV vector further comprises a cGAS polynucleotide (cyclic GMP-AMP synthase polynucleotide) which is a third template for synthesis of a cGAS protein, where the cGAS protein is constitutively active.

17. The method of claim 14, where the mammal is a human subject and the mammalian STING polynucleotide is human STING polynucleotide, where the human subject is suffering from a cancerous tumor, where the cancerous tumor is selected from the group consisting of an ovarian cancer tumor, a colon cancer tumor, a melanoma tumor, a breast cancer tumor, and a lung cancer tumor.

18. The method of claim 14, further comprising administering an adjuvant.

19. The method of claim 14, where the recombinant VSV vector is administered intratumorally, intravenously, intra-arterially, intraperitoneally, intranasally, intramuscularly, intradermally or subcutaneously to the host cell.

20. The method of claim 14, where the PEG-ated-IFN is administered intratumorally, intravenously, intra-arterially, intraperitoneally, intranasally, intramuscularly, intradermally or subcutaneously to the mammal.


SEQUENCE LISTING (17 sequences)

SEQ ID NO: 1 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 2 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 3 - DNA, 33 bp, synthetic construct
  misc_feature 1..33: note = Synthetic Primer
  misc_feature 1..33: note = hSTING_R284S-F
  source 1..33: mol_type = other DNA
tttagccgggaggattctcttgagcaggccaaa

SEQ ID NO: 4 - DNA, 33 bp, synthetic construct
  misc_feature 1..33: note = Synthetic Primer
  misc_feature 1..33: note = hSTING_R284S-R
  source 1..33: mol_type = other DNA
tttggcctgctcaagagaatcctcccggctaaa

SEQ ID NO: 5 - DNA, 28 bp, synthetic construct
  misc_feature 1..28: note = Synthetic Primer
  source 1..28: mol_type = other DNA
ctagctcgagatgccccactccagcctg

SEQ ID NO: 6 - DNA, 28 bp, synthetic construct
  misc_feature 1..28: note = Synthetic Primer
  source 1..28: mol_type = other DNA
ctaggctagctcaagagaaatccgtgcg

SEQ ID NO: 7 - DNA, 66 bp, synthetic construct
  misc_feature 1..66: note = Synthetic Primer
  source 1..66: mol_type = other DNA
ggaagcggagctactaacttcagcctgctgaagcaggctggagacgtggaggagaaccctggacct

SEQ ID NO: 8 - DNA, 31 bp, synthetic construct
  misc_feature 1..31: note = Synthetic Primer
  misc_feature 1..31: note = pTransfer-STING forward
  source 1..31: mol_type = other DNA
gatcactagtatgccccactccagcctgcat

SEQ ID NO: 9 - DNA, 34 bp, synthetic construct
  misc_feature 1..34: note = Synthetic Primer
  misc_feature 1..34: note = pTransfer-STING reverse
  source 1..34: mol_type = other DNA
gatcctcgagtcaagagaaatccgtgcggagagg

SEQ ID NO: 10 - DNA, 34 bp, synthetic construct
  misc_feature 1..34: note = Synthetic Primer
  misc_feature 1..34: note = pTransfer-cGAS forward
  source 1..34: mol_type = other DNA
gatcactagtatgcagccttggcacggaaaggcc

SEQ ID NO: 11 - DNA, 31 bp, synthetic construct
  misc_feature 1..31: note = Synthetic Primer
  misc_feature 1..31: note = pTransfer-cGAS reverse
  source 1..31: mol_type = other DNA
gatcctcgagtcaaaattcatcaaaaactgg

SEQ ID NO: 12 - DNA, 31 bp, synthetic construct; identical to SEQ ID NO: 8
  misc_feature 1..31: note = Synthetic Primer
  misc_feature 1..31: note = pTransfer-STING-2A-cGAS 1st step PCR forward
  source 1..31: mol_type = other DNA

SEQ ID NO: 13 - DNA, 68 bp, synthetic construct
  misc_feature 1..68: note = Synthetic Primer
  misc_feature 1..68: note = pTransfer-STING-2A-cGAS 1st step PCR reverse
  source 1..68: mol_type = other DNA
ctccacgtctccagcctgcttcagcaggctgaagttagtagctccgcttccagagaaatccgtgcgga

SEQ ID NO: 14 - DNA, 83 bp, synthetic construct
  misc_feature 1..83: note = Synthetic Primer
  misc_feature 1..83: note = pTransfer-STING-2A-cGAS 2nd step PCR forward
  source 1..83: mol_type = other DNA
ggaagcggagctactaacttcagcctgctgaagcaggctggagacgtggaggagaaccctggacctatgcagccttggcacgg

SEQ ID NO: 15 - DNA, 31 bp, synthetic construct; identical to SEQ ID NO: 11
  misc_feature 1..31: note = Synthetic Primer
  misc_feature 1..31: note = pTransfer-STING-2A-cGAS 2nd step PCR reverse
  source 1..31: mol_type = other DNA

SEQ ID NO: 16 - DNA, 31 bp, synthetic construct; identical to SEQ ID NO: 8
  misc_feature 1..31: note = Synthetic Primer
  misc_feature 1..31: note = pTransfer-STING-2A-cGAS 3rd step PCR forward
  source 1..31: mol_type = other DNA

SEQ ID NO: 17 - DNA, 31 bp, synthetic construct; identical to SEQ ID NO: 11
  misc_feature 1..31: note = Synthetic Primer
  misc_feature 1..31: note = pTransfer-STING-2A-cGAS 3rd step PCR reverse
  source 1..31: mol_type = other DNA